(19) United States
(12) Patent Application Publication
(10) Pub. No.: US 2012/0042882 A1
(43) Pub. Date: Feb. 23, 2012

(54) PARTICLES WITH CHARGED SURFACE DOMAINS

(75) Inventor: Carolina Salvador Morales, Brookline, MA (US)

(73) Assignee: Massachusetts Institute of Technology, Cambridge, MA (US)

(21) Appl. No.: 12/861,781

(22) Filed: Aug. 23, 2010

(51) Int. Cl. A61B 19/00; C09B 67/02

(52) U.S. Cl. 128/898; 8/638; 8/636

(57) ABSTRACT

Methods include forming an adaptive aesthetic marking, such as a tattoo that is at least occasionally visible on a surface of skin by introducing, into a layer of skin, a plurality of particles, each comprising a core and a surface domain. The surface domain comprises a net charge and the core or surface domain, or both, includes a dye. A particle includes a core, first linkers and second linkers. Each first linker comprises a first end that binds to the core, and a second end that comprises a first functional group having a first charge. Each second linker comprises a first end that binds to the core, and a second end that comprises a second functional group having a different second charge. The first and second functional groups form an external mosaic of surface domains, each domain comprising a majority of one type of functional group.

### BACKGROUND OF THE INVENTION

Spherical colloidal particles are ubiquitous in drug delivery, in vivo and in vitro diagnostics, as well as additives in almost every industry (food, cosmetics, paints, etc). The ability of these particles to accurately interact with biological organisms, cells and molecules in a complex mixture or in vivo is crucial in both basic research and clinical settings. The vast majority of particles used in suspension arrays are optically encoded latex microspheres with diameters between 0.3 and 10 microns (1 micron=10⁻⁶ meters) that can be interrogated and decoded with laser-based flow cytometry (measurement of cell sized particles). Optical encoding is accomplished by swelling the spheres with fluorescent organic dyes with different emission spectra. While recent advances in the field of colloid synthesis have produced non-spherical particles, the ability to impart electronic charge density domains on portions of a single particle and to determine uses for such particles have not been demonstrated.

### SUMMARY OF THE INVENTION

Techniques are provided for particles functionalized at least with one or more charged surface domains, such as can be used in adaptive aesthetic markings including reversible tattoos.

In a first set of embodiments, a particle comprises a core structure having a surface, a plurality of first linkers and a plurality of second linkers. Each of the first linkers includes a first end that binds to the surface of the core structure and a second end that includes a first functional group that has a first charge. Each of the second linkers includes a first end that binds to the surface of the core structure and a second end that includes a second functional group that has a different second charge.

In another set of embodiments, a method comprises forming a tattoo that is at least occassionally visible on a surface of skin by introducing, into a layer of the skin, a plurality of particles. Each particle includes a core structure and at least one surface domain that comprises a net charge. At least one of the core structure or the surface domain further comprises a dye.

In some of these embodiments, the method also includes placing an electrode near a surface of the skin and applying an electric field through the electrode sufficient to cause the plurality of particles to move from the layer of skin toward the electrode.

In another set of embodiments, a method comprises forming a tattoo that is at least occasionally visible on a surface of skin by introducing, into a layer of the skin, a plurality of particles. Each particle comprises a core structure and a first surface domain and a different second surface domain. The first surface domain comprises a first dye and a first net charge. The second surface domain comprises a different second net charge. The first dye is substantively absent in the core structure and in the second surface domain.

In some of these embodiments, the method also includes placing an electrode near a surface of the skin and applying an electric field through the electrode sufficient to cause the plurality of particles to orient in the skin based on the electric field.

In another set of embodiments, a particle comprises a core structure and a first surface domain and a different second surface domain. The first surface domain comprises a first dye and a first net charge. The second surface domain comprises a different second net charge. The first dye is substantively absent in the core structure and in the second surface domain.

Still other aspects, features, and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1A is a block diagram that illustrates an example particle with a surface domain different from a core structure, according to an embodiment;

FIG. 1B is a block diagram that illustrates an example particle with two different surface domains, each different from a core structure, according to an embodiment;

FIG. 1C is a block diagram that illustrates an example particle with multiple surface domains, each different from a core structure, according to an embodiment;

FIG. 2 is a flow diagram that illustrates an example method for making and using particles with at least one charged surface domain, according to an embodiment;

FIG. 3A is a micrograph that illustrates an example population of particles that includes particles with at least one charged surface domain, according to an embodiment;

FIG. 3B is a micrograph that illustrates an example population of particles in which one surface domain is randomly distributed over the particles' surfaces, according to an embodiment;

FIG. 3C is a micrograph that illustrates an example population of particles that includes particles with at least two charged surface domain, according to an embodiment;

FIG. 3D. is a micrograph that illustrates an example population of particles in which one surface domain is randomly distributed over the microparticles' surfaces, according to an embodiment;

FIG. 4A is a block diagram that illustrates an example color observed from a population of randomly oriented particles that each include two surface domains of differently net charge and different dyes, according to an embodiment;

FIG. 4B is a block diagram that illustrates an example color observed from a population of particles of FIG. 4A in a first electric field, according to an embodiment;

FIG. 4C is a block diagram that illustrates an example color observed from a population of particles of FIG. 4A in a second electric field perpendicular to the first electric field, according to an embodiment;

FIG. 4D is a block diagram that illustrates an example color observed from a population of particles of FIG. 4A in a third electric field opposite to the second electric field, according to an embodiment;

FIG. 5 is a block diagram that illustrates an example range of colors observed in an electric field from a population of particles that each include two different surface domains but of different total charges, according to an embodiment;

FIG. 6A is a block diagram that illustrates an example color observed from a population of particles that each include two different surface domains in an electric field of a first strength;

FIG. 6B is a block diagram that illustrates an example color observed from a population of particles that each include two different surface domains in an electric field of a second strength;

FIG. 7A is a block diagram that illustrates an example particle that includes two surface domains of differently net charge and different dyes and a core structure that includes a third dye, according to an embodiment;

FIG. 7B is a block diagram that illustrates an example tattoo comprising a population of the particles of FIG. 7A, according to an embodiment;

FIG. 7C is a block diagram that illustrates an example tattoo comprising a population of the particles of FIG. 7A in the presence of an electric field, according to an embodiment;

FIGS. 8A through 8G are photographs that illustrates colors observed from a population of the particle of FIG. 7A before and after applying an electric field, according to various embodiments;

FIG. 9A is a block diagram that illustrates an example particle that includes a single surface domain of negative net charge and blue dye, according to an embodiment;

FIG. 9B is a block diagram that illustrates an example tattoo comprising a population of the particles of FIG. 9A, according to an embodiment;

FIG. 9C is a block diagram that illustrates an example tattoo comprising a population of the particles of FIG. 9A after application of an electric field, according to an embodiment;

FIGS. 10A through 10H are photographs that illustrate example migration of the particles of FIG. 9A after applying an electric field, according to various embodiments;

FIGS. 11A through 11C are photographs that illustrate example deposition of the particles of FIG. 9A on a positive electrode, according to an embodiment;

FIGS. 12A through 12F are photographs that illustrate example removal of the particles of FIG. 9A after applying an electric field using adhesive electrodes, according to various embodiments; and

FIG. 13 is a micrograph that illustrates example duration of particles after six months in an aqueous environment indicative of biodegradability, according to an embodiment.

### DETAILED DESCRIPTION

A method and composition of matter are described for particles with one or more charged surface domains, such as for reversible tattoos. In some embodiments, one or more such charged domains are also functionalized with additional functions, such as with dyes and chemicals. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Some embodiments of the invention are described below in the context of particles with at least one colored surface domain to be injected into skin for a tattoo. However, the invention is not limited to this context. In other embodiments particles with one or more differently charged surface domains are used for other purposes, with or without dyes, including safe, painless, instantaneously or temporary removable color tattoos, flexible displays, cosmetic surgery, electronic paint for fashion and fabrics, adaptive camouflage, smart card, logos on products, optogenetics, delivery of compounds for wound healing, cardiology and pain medicine, medical circuits, protein transistors, medical tattoos (e.g., for radiotherapy), paint for artists, performer's clothing, smart cards for army applications, or voltage gates in neurosciences, among others, alone or in any combination.

### 1. Overview

The formation of multiple functional domain particles has been described elsewhere, e.g., in international patent application WO/2010/042555, the entire contents of which are hereby incorporated by reference as if fully set forth herein, except for terminology inconsistent with that defined herein. As described therein, the formation of such particles includes obtaining a core structure having a surface and a plurality of first linkers and a plurality of second linkers. Each first linker includes a first end that binds to the surface of the core structure, and a second end that has a first functional group. Each second linker includes a first end that binds to the surface of the core structure, and a second end that includes a second functional group, different than the first. The first ends of the linkers are bound to the surface of the core structure via their respective first ends. The first and second functional groups form an external mosaic of surface domains, each domain including a majority of one type of functional group.

For example, a polymeric nanoparticle having multiple functionalized surface domains are formed by dissolving a polymer in a volatile, water-miscible organic solvent to form a first solution; dissolving a plurality of first and second amphiphilic components bound to linkers in an aqueous solvent to form a second solution. The amphiphilic components each include a hydrophobic end and a hydrophilic end. The first linkers each include a first functional group, and the second linkers each include a second functional group. The first and second solutions are combined such that a polymeric nanoparticle is formed having a polymer core surrounded by the amphiphilic components. The linkers extend from the amphiphilic components and the first and second functional groups form an external mosaic of surface domains, each domain including a majority of one type of functional group.

In these methods, the first and second linkers can be the same except for the different functional groups, and the methods can further include obtaining a plurality of third (or fourth or more) linkers, different from the first and second linkers, and binding first ends of the third linkers to the surface of the core structure. In certain embodiments, the final particles can have mosaic patterns that include two surface domains, a first domain including a majority of the first functional groups, and a second domain including a majority of the second functional groups. In other embodiments, the mosaic pattern can have multiple surface domains each including a majority of first functional groups and multiple surface domains each including a majority of second functional groups.

According to various embodiments of the present invention, at least one of the functional groups includes a net charge. The charge density (charge per unit area) in each surface domain depends on the mix of first and second linkers in each surface domain. The net charge of the particle depends on the charge density and the size of each domain and any charge in the core structure.

In some of these embodiments, the charged functional group also includes a dye. As used herein, a dye includes any source of color, including any chromophore, dye, fluorescent protein, or other material that emits light of a limited wavelength range under at least some conditions, alone or in some combination.

In some embodiments, the dyes and other component materials of the particles are selected from among materials that have been approved by the United States Food and Drug Administration for use in human.

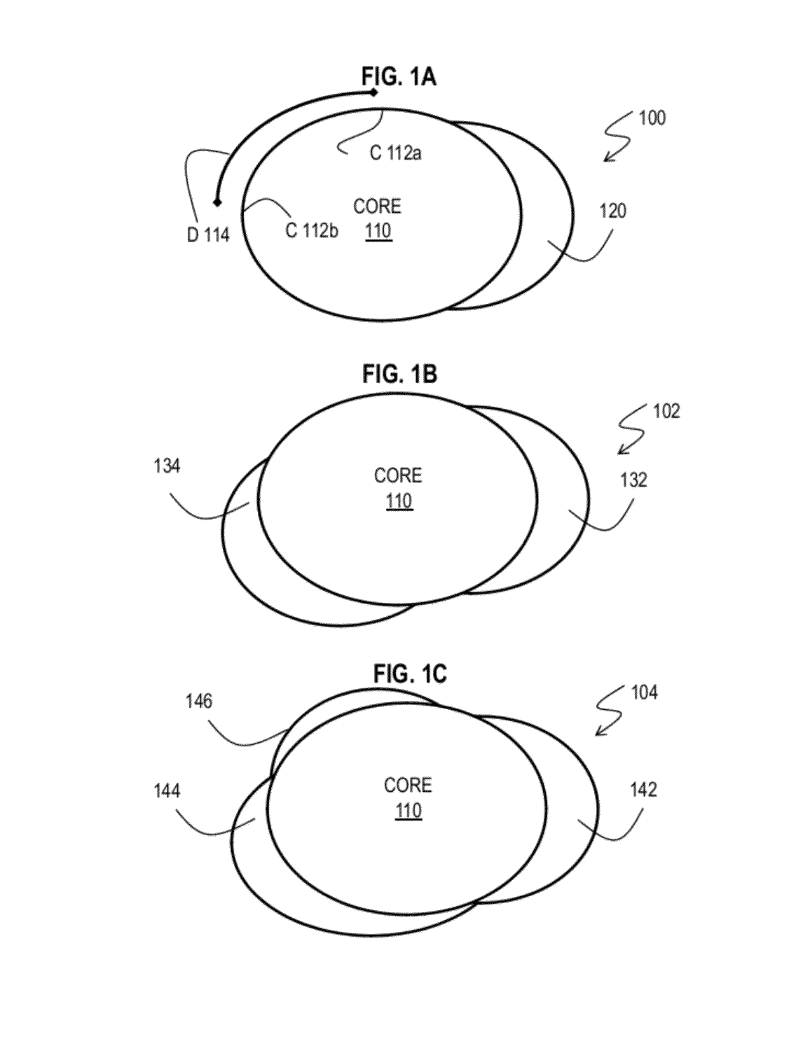

FIG. 1A is a block diagram that illustrates an example particle 100 with a surface domain 120 different from a core structure 110, according to an embodiment. The surface domain 120 comprises a plurality of first linkers with a first end bound to the core structure 110 and a second end that comprises a first functional group having a first charge. As a result of the net charge per first functional group, the surface domain 120 has a net charge density per unit area. The global charge of the particle's surface depends on the magnitude of the charge of the particle's surface domains and the charge, if any, on the core structure.

The core structure 110 has a minimum curvature (maximum radius of curvature) of C 112a at one location, and has a maximum curvature (minimum radius of curvature) of C 112b at a second location, separated from the location of minimum curvature by a distance d 114. For spherical particles C 112a=C 112b; and d=2πr/4, where r is the radius of the sphere. Any material may be used as the core structure 110, including polymeric, metal and core-shell particles as well as reverse micelles, metal oxide, and quantum dots.

A particle 100 with a single surface domain 120 can be synthesized in any manner known in the art, including nanoprecipitation (also known as solvent displacement), emulsification-solvent evaporation, thin-film hydration, modified Stober method, layer-by-layer synthesis, ionotropic gelification, ultrasonication, and polymer-monomer pair reaction. In the illustrated embodiment, the surface domain 120 has a charge density that depends on the net charge of each functional group on each linker. In some embodiments, a dye is included in the functional group of the linkers in surface domain 120, or in the core structure 110, or both.

FIG. 1B is a block diagram that illustrates an example particle 102 with two different surface domains 132 and 134, each different from a core structure 110, according to an embodiment. The number of different surface domains depends on the number of different components exposed to the core structure 110 during synthesis. In some embodiments, each of surface domain 132 and surface domain 134, collectively referenced hereinafter as surface domains 130, comprises one type of linker with one type of functional group. In some embodiments, each of surface domains 130 includes a different ratio of two or more linkers with associated functional groups. In some embodiments, one or more of the functional groups has a net charge. The charge density in each surface domain 130 depends on the net charge of each functional group and the ratio of the two or more functional groups in each surface domain. In some embodiments, a dye is included in the functional group of one or more of the linkers, or in the core structure 110, or some combination; and the particle 102 is at least partially colored.

FIG. 1C is a block diagram that illustrates an example particle 104 with multiple surface domains 142, 144, 146, each different from a core structure 110, according to an embodiment. In some embodiments, each of surface domain 142 and surface domain 144 and surface domain 146, collectively referenced hereinafter as surface domains 140, comprises one type of linker with one type of functional group from a set of three or more functional groups available during synthesis. In some embodiments, each of surface domains 140 includes a different ratio of two or more linkers with associated functional groups. In some embodiments, one or more of the functional groups has a net charge. The charge density in each surface domain 140 depends on the net charge of each functional group and the ratio of the two or more functional groups in each surface domain. In some embodiments, a dye is included in the functional group of one or more of the linkers, or in the core structure 110, or some combination; and the particle 104 is at least partially colored.

The global charge of any of the particles 100, 102 or 104 is tunable by the selection of the net charge per functional group. Furthermore, in some embodiments, the charge of the particle is increased by adding sodium dodecyl sulfate (SDS) or hexadecyltrimethylammoniumbromide (CTBA), or some combination. To increase the charge of a particle's surface, SDS (negatively charged) or CTBA (positively charged) is added after the synthesis of the particles. In some embodiments, the magnitude of the particle's charge is modulated further by varying the molar ratio between pegylated lipids and the SDS or CTBA. The ability to manipulate the charge density of the microparticles allows control of the electrophoretic mobility of the particles. The better the electrophoretic mobility, the faster the response to an external electric field, e.g., for color formation or color switching capability of the particles.

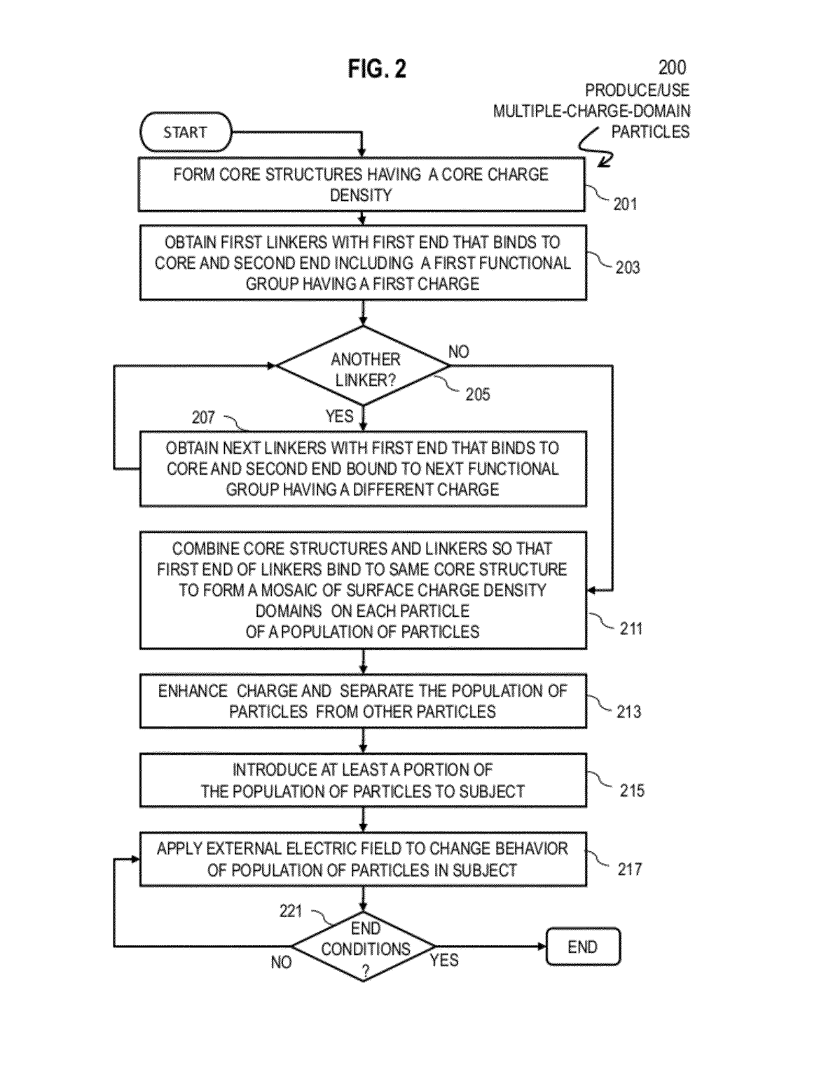

FIG. 2 is a flow diagram that illustrates an example method for making and using particles with at least one charged surface domain, according to an embodiment. Although steps are depicted in FIG. 2 as integral blocks in a particular order for purposes of illustration, in other embodiments, one or more steps, or portions thereof, may be performed in a different order, or overlapping in time, in series or in parallel, or one or more steps, or portions thereof may be omitted, or additional steps added, or the method may be changed in some combination of ways. For example, in some embodiments, steps 201, 203 and zero or more passes of step 207 are performed during the same time interval.

In step 201, core structures that have a core charge density are formed. For example, one or more monomer components of the core structure are placed in solution and polymerized using any method known in the art. In some embodiments, the core structures are formed simultaneously with the surface domains, so that step 201 overlaps step 211, described below. In some embodiments, the core structure 110 is electrically neutral so that the core charge density is zero. In some embodiments, the core structure 110 includes a dye. In some embodiments, the core particle is a microparticle with a maximum dimension no greater than 1000 micrometers (μm, also called microns, 1 micron=10⁻⁶ meters). In some embodiments, the core particle is a nanoparticle with a maximum dimension no greater than 1000 nanometers (nm, 1 nm=10⁻⁹ meters). In some embodiments, the core structure is biodegradable.

In step 203 first linkers are obtained, each with a first end that binds to the core structure and a second end that includes a first functional group having a first charge. In some embodiments, the first functional group is electrically neutral so that the first charge is zero. More detail on linkers and functional groups are described below with reference to particular embodiments.

In the illustrated embodiments, the charge density of a surface domain is characterized by a Zeta potential of a particle including that surface domain. A Zeta potential is the electrical potential difference between a dispersion medium and the stationary layer of fluid attached to a dispersed particle. A positive or negative value of 25 millivolts (mV, 1 mV=10⁻³ Volts) is taken as an arbitrary value that separates low-charged surfaces from highly-charged surfaces. Zeta potential is not measurable directly but it can be calculated using theoretical models and an experimentally-determined electrophoretic mobility or dynamic electrophoretic mobility. Electrophoresis is used for estimating zeta potential of particulates, as is well known in the art.

All zeta potential measurements recited herein were performed using a Malvern Instrument of Westborough Mass. A linker component, such as a pegylated lipid functional group, dyes (red fluorescent, blue fluorescent) or colored pegylated lipid functional group, was suspended in 4% ethanol and placed in a folded capillary cell (disposable electrode). The Smoluchwski equation, given by Equation 1 was used to extract the zeta potential, ζ, from the measured particle electrophoretic mobility,

ζ=4π82 η/D (1)

where μ, η and D are the electrophoretic mobility, viscosity and dielectric constant of the dispersion medium, respectively. The reported zeta potential values are an average of 3 measurements, each of which was obtained over 20 electric field cycles. The effective voltage was 151 Volts (V).

For example, some linkers include 1, 2-Distearoyl-sn-Glycero-3-Phosphoethanolamine-N-Polyethylene Glycol (DSPE-PEG) at a first end with a second end chosen from amine (NH₂) or maleimide (MAL). In some embodiments, a red fluorescent dye (PKH26 Red Fluorescent Cell Linker) was included in the amine functional group; and a blue fluorescent dye was included in the maleimide functional group. The formations of such particles are described in more detail below with reference to particular embodiments. Table 1 depicts the measured Zeta potentials of particles with a surface domain comprising the linker components listed in the Table.

TABLE 1
Charge density of each of the linkers that form example
charged microparticles

| Linker component | Zeta potential (mV) |
|---|---|
| DSPE-PEG-MAL | No detectable |
| DSPE-PEG-NH₂ | No detectable |
| Red fluorescent | +47 ± 2 |
| Blue fluorescent | −10 ± 2 |
| DSPE-PEG-MAL-Blue fluorescent | −31 ± 3 |
| DSPE-PEG-NH₂₋red fluorescent | 16 ± 2 |

Thus in these embodiments, the dye contributes to the net charge provided by each linker. Other dyes and functional groups that provide net charges in various embodiments include Fluorescein Isothiocyanate (FICT), Alexa fluor 488, Alexa fluor 633, FITC-dextran, Rhodamine-B-dextran, SE (molecular probes), Alexa 647, Green Fluorescent Nucleic Acid Stain, Red Fluorescent Nucleic Acid Stain, Blue Fluorescent Nucleic Acid Stain, Orange Fluorescent Nucleic Acid Stain, Bodipy fluorescein (Bodipy FL), Bodipy rhodamine 6G (Bodipy R6G), Bodipy tetramethylrhodamine (Bodipy TMR), Bodipy texasRed fluorophores (Bodipy TR), Amine reactive bodipy dyes, Thiol-reactive bodipy dyes Bodipy succinimidyl esters, water-soluble bodipy sulfonated succinimidyl esters, bodipy carboxylic acids, bodipy lipids or bodipy receptor ligand conjugate, alone or in some combination. The following functional groups contribute to the net charge provided by each linker, in various embodiments: amine, maleimide, hydroxyl, carboxyl, methoxyl, pyridythiol, azide group, hydrocarbyls, ketone, aldehyde, acyl halide, alcohol, carbonate, carboxylate, carboxylic acid, ether, ester, hydroperoxide, peroxide, amide, amine, imine, imide, azo compounds, cyanates, isocyanates, nitrate, nitrile, nitrite, nitrocompound, nitroso compound, or pyridine derivatives, alone or in some combination.

In step 205, it is determined whether there is another linker to be used in the synthesis of particles. If so, then in step 207, similar to step 203, next linkers are obtained, each with a first end that binds to the core structure and a second end that includes a next functional group having a next charge different from the first charge. In some embodiments in which the first net charge is non-zero, the next functional group is electrically neutral so that the next charge is zero. Steps 205 and 207 form a loop that is repeated until all linkers to be used in the synthesis of a population of particles are obtained.

If it is determined in step 205 that there is not another linker to be included during the synthesis, then in step 211, the core structures and linkers are combined so that the first ends of multiple linkers from one or more of all the types of linkers bind to the core structure to form a mosaic of one or more surface charge density domains on each particle of a population of particles.

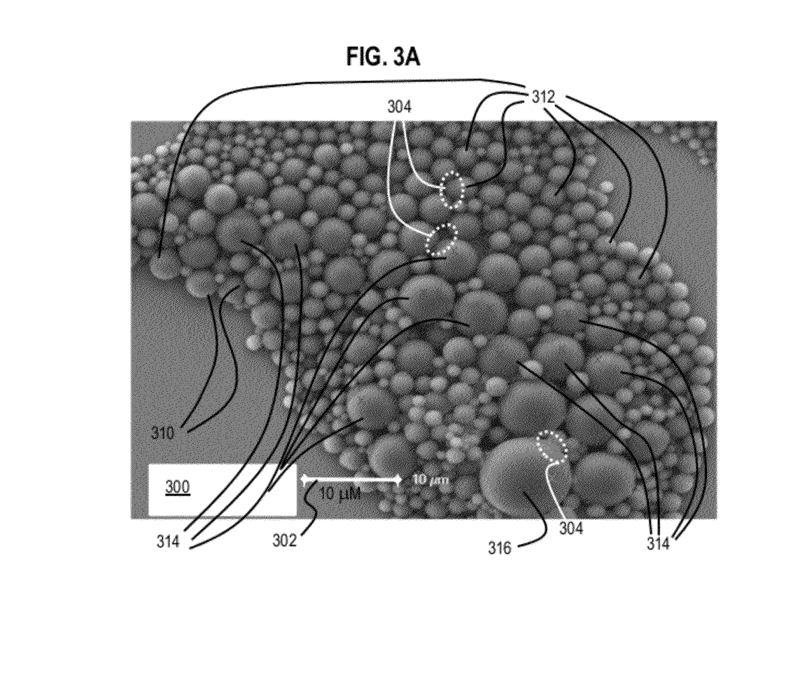

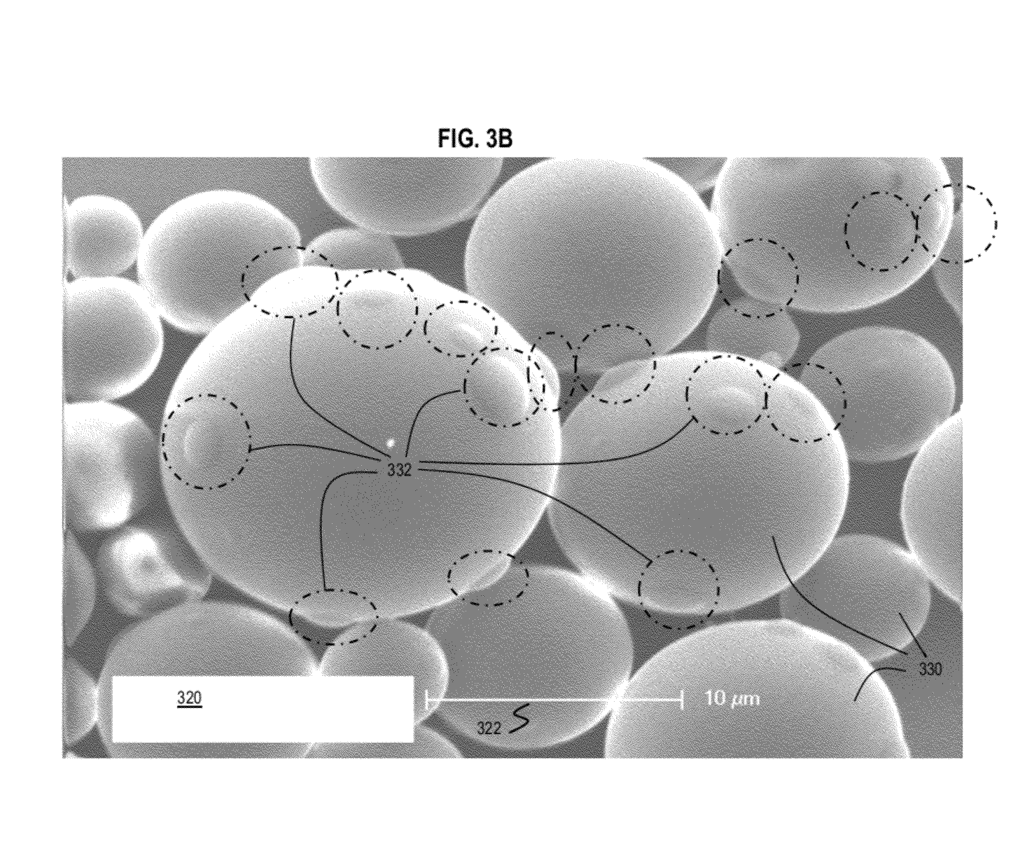

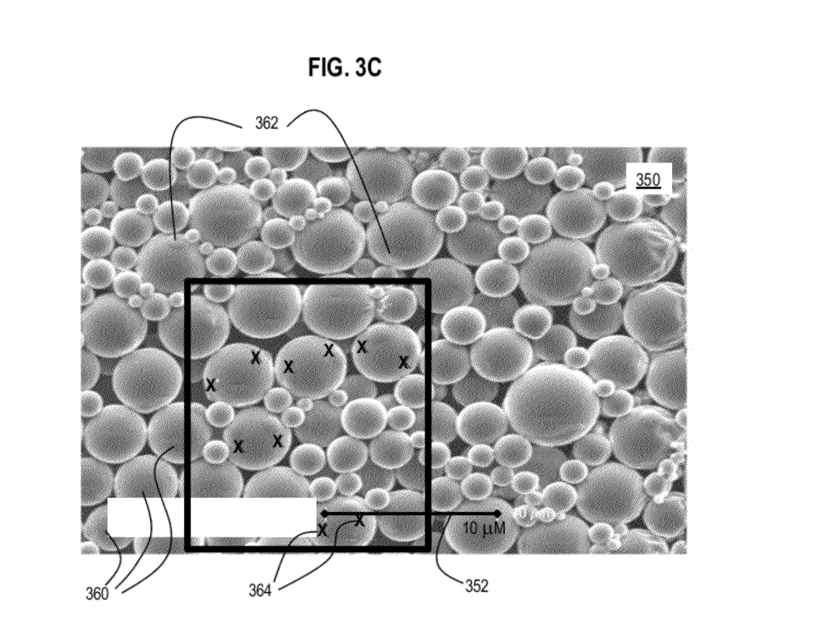

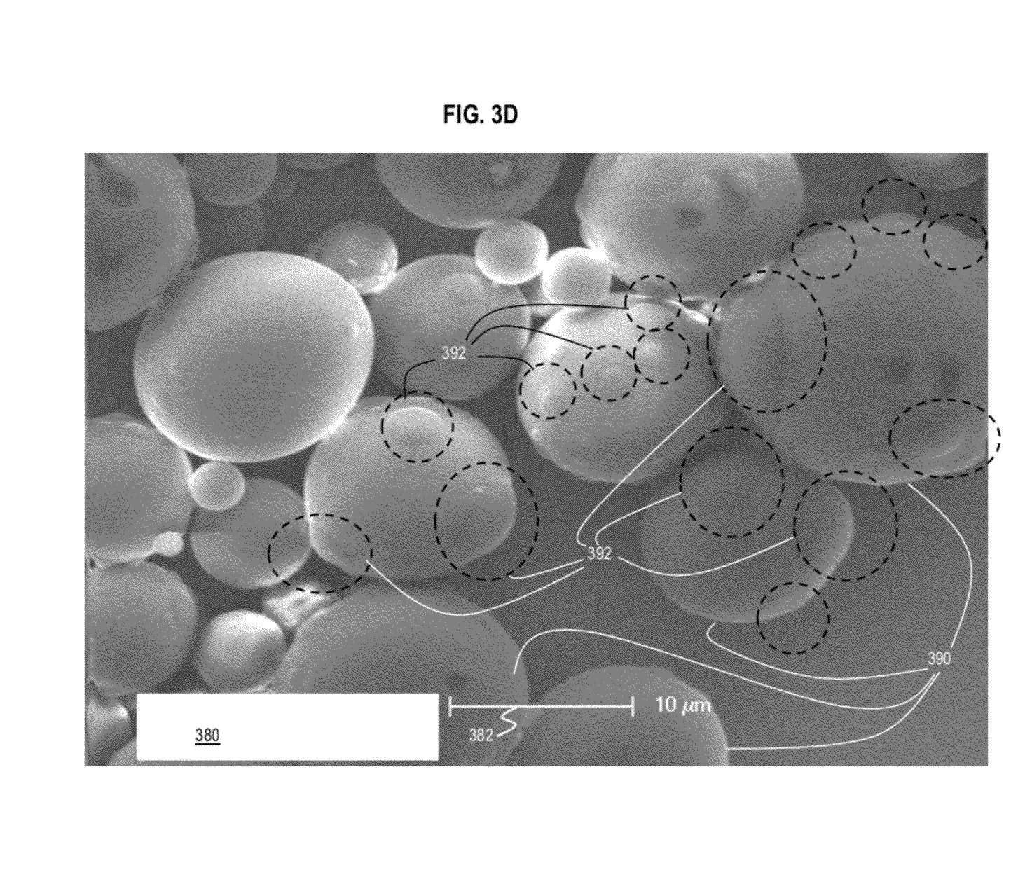

FIG. 3A is a micrograph 300 that illustrates an example population of particles 310 that includes particles with at least one charged surface domain, according to an embodiment. The micrograph was obtained from a scanning electron microscope of particles functionalized with 50% DSPE-PEG-NH₂(+) and 50% DSPE-PEG-MAL-blue fluorescent dye(−). The micrograph scale 302 depicts 10 microns. The population of particles 310 includes both microparticles and nanoparticles. Surface domains 304 on three particles are indicated. The population of particles 310 includes particles 312 of relatively high curvature and small size, and particles 314 of moderate curvature and size, and particles 316 of low curvature and large size.

FIG. 3B is a micrograph 320 that illustrates an example population of particles 330 in which one surface domain 332 is randomly distributed over the particle's surface, according to an embodiment. The micrograph scale 332 depicts 10 microns. Some example surface domains 332 are surrounded by dot-dashed ovals. These polymer mciroparticles 330 were prepared by functionalizing PLGA with 100% DSPE-PEG-NH₂ using the emulsion method as described in the next section.

FIG. 3C is a micrograph 350 that illustrates an example population of particles 360 that includes particles with at least two charged surface domain, according to an embodiment. The micrograph was obtained from a scanning electron microscope of particles functionalized with 50% DSPE-PEG-NH₂-red fluorescent dye(+) and 50% DSPE-PEG-MAL-blue fluorescent dye(−). The micrograph scale 352 depicts 10 microns. The population of particles 360 includes both microparticles and nanoparticles. Multiple surface domains 364 on some five particles inside a bounding square are indicated by a pair of Xs. Outside of the bounding square there are many other particles showing at least one visible domain 362.

FIG. 3D. is a micrograph 380 that illustrates an example population of particles 390 that have multiple charged surface domains 392 that are randomly distributed over the microparticles' surfaces, according to an embodiment. The micrograph scale 382 depicts 10 microns. The micrograph 380 was obtained from a scanning electron microscope of particles functionalized with 100% DSPE-PEG-MAL-Blue fluorescent dye(−). These polymer microparticles were prepared by functionalizing PLGA with 100% DSPE-PEG-MAL using the emulsion method as described in the next section.

Returning to FIG. 2, in step 213, the charge in one or more functional groups is enhanced, e.g., by adding SDS or CTBA, or some combination. Table 2 depicts the effects of enhancing the charge on particles in two embodiments.

TABLE 2
Manipulating a particle's charge by adding SDS or CTBA

| | Zeta potential of | Zeta potential of |
|---|---|---|
| | particles without | particles after |
| | adding SDS or | adding SDS or |
| | CTBA to particle | CTBA to particle |
| linker | preparation (mV) | preparation (mV) |
| 100% DSPE-PEG-MAL | −25 ± 2 | −113 ± 2 (SDS) |
| 100% DSPE-PEG-NH₂ | 5 ± 1 | 63 ± 3 (CTBA) |

In some embodiments, step 213 includes separating the particles in the population, e.g., by total charge or number of surface domains or size or weight or some combination. In some embodiments, step 213 is omitted.

In step 215, at least a portion of the population of particles is introduced to a subject. For example, the particles are used as ink on paper, or food, or in tattoos applied to the skin of a subject. In various embodiments, the particles are introduced to other subjects for other applications, e.g., for air filter applications through medicinal camouflage applications, as described below.

In step 217, an electric field is applied in the vicinity of the subject to change the behavior of the portion of particles introduced to the subject. For example, the electric field is applied to remove the particles (e.g., during removal of a tattoo) or to separate the particles with different net charges (e.g., to separate differently colored particles) or to orient the particles with non uniform charge densities (e.g., to change the perceived color at a fixed location), or to rotate in a 3D space, or some combination. Such functionality is particular advantageous for imaging applications especially when charged surface domains are envisaged to be used as a Foster Resonance Energy Transfer System as described below.

In step 221, it is determined if end conditions are satisfied, e.g., that the desired behavior has been achieved (such as removing the tattoo). If not, the process returns to step 217 to again apply an electric field. Otherwise, the process ends.

Air filter applications. In some embodiments, charged surface domain particles are used to create a paint that coats a wide range of products to retain dust or other toxic materials via an electrostatic interaction. For example, it is known that facial protective masks are not efficient enough to prevent inhaling nanomaterials such as carbon nanotubes and other nanoparticles. Coating these masks with a paint made of charged surface domain particles diminishes this problem. Furthermore, by applying an electric field on these masks, carbon nanoparticles are removed from the masks; therefore the mask can be reused. This represents a mechanism to prevent and diminish the toxic effects of such nanomaterials as well as providing an environmentally friendly procedure. In various embodiments, this technology is applied to many clean wipe systems. The fact that multiple charged surface domains can have different surface chemistry allows customization of the chemical composition of the filters in various embodiments, according to the type of particles that are to be filtered. For instance, in some embodiments, a filter is placed in the human nose to prevent bacteria or virus from entering respiratory airways. Many bacteria and virus have a layer of carbohydrates on their surfaces. The property that the dual charged surface domains are functionalized virtually by any functional groups is utilized in various embodiments to prevent respiratory diseases caused by many pathogens that have different surface chemistries.

Anti-counterfeit applications. To date, there are several devices for detecting counterfeiting, including ultraviolet light-emitting diode (UV-LED) counterfeit detectors. Although these systems are efficient, one of the biggest drawbacks of these technologies is the harmful effect that UV can produce in users' eyes. The use of charged surface multiple domain particles offers the possibility to create counterfeiting detection devices by using particles that have one or two domains which are colored and charged. In various embodiments, these particles are incorporated in a tag that displays a visible color in the presence of an electric field and becomes uncolored when the electric field changes. For example, in some embodiments, only in the presence of an electric field does the color of the particle appear. The use of an electric field which displays the particle's color represents an easy, intuitive design, offering cheap, efficient and instant visual detection. Most importantly, such anti-counterfeit embodiments are not harmful to the users' eyes. Also, another major advantage of some such embodiments is simplicity, because hundreds of code sequences, registration and central database storage are not required.

Adaptive fashions. In some embodiments, such charged and colored particles are placed on different types of fabrics including cotton, for example, by using a tattoo machine. In some embodiments, the multiple domains or cores of the particles are made of dyes which are sensitive to light, as described below, then the color of the fabric changes under different lighting conditions. Also, by applying an electric field to the fabric one can modulate the strength of the color that is displayed. This means that, in some embodiments, a user can change the color of the fabric by changing the properties of the electric field, such as the magnitude of the voltage. This technology is applied to any type of clothing in various embodiments. For example, in some embodiments, shoes are painted with a color electronic ink that changes colors by applying an electric field.

Adaptive adhesives. In some embodiments, charged domain particles are used for the creation of adhesives with different magnitudes of adhesive strength. The adhesive is composed of particles with charged multiple domains. Since these domains have been charged, they are attached to the surface via an electrostatic interaction. The strength of the electrostatic interaction is regulated by the electric field. Also, in some embodiments, changes in magnitude of the electric field's strength are identified by a change of the particle's color. The visible color allows proper alignment of components before bonding. The increase of the adhesive strength is controlled by the magnitude of the electric field. In some embodiments, this procedure is less harmful to a user than the use of a UV lamp for affecting adhesion, making the procedure safe for medical applications, such as for patients that undergo surgery. In various embodiments, these adhesives are clean, easy and practical, in any combination.

Adaptive art. In some embodiments, a color electronic ink is used for paintings. Because electronic ink embodiments are made of particles with colored and charged surface domains, one can change the color of the ink by changing the properties of the electric field. Using this technology a painter can make local or global changes in a painting.

Instantaneously removable logos. Instantaneously removable logos are created using an electronic ink made of particles that have charged surfaces domains. Only one domain is colored, the other domain is uncolored. By applying an electric field, the domain that is colored will become visible or invisible as desired. This procedure saves money and time so that a logo visible on a product in the market is invisible during use of the product.

Protein transistors. In some embodiments, charged surface domains particle are used to bridge two metal electrodes. In some embodiments, these particles are synthesized to include a lipid bilayer. Embedded in that layer is an Adenosine-5′-triphosphate (ATP)-powered pump, which is widespread in cells and which mediates the exchange of sodium and potassium ions across membranes. The central part of the charged surface domain particles is exposed to an ATP solution. When the device is switched on, the pump pushes ions across the lipid membrane, changing the conductance of the lipid particle and boosting the transistor's current output.

Adaptive cell imaging. In some embodiments, charged surface domain particles are used as a double or multiple fluorescence imaging tool to investigate the nature of the conformational changes that are associated with substrate binding and transport phenomena within the cell. For example, in some embodiments, particles each with multiple charged surface domains are used to observe and quantify time-dependent changes in biological structures which could be masked by ensemble averaging in bulk measurements or suppressed during crystallographic conditions. In such embodiments, these particles work precisely like Forster resonance energy transfer (FRET) molecules by incorporating Cy3 and Cy5 in one of the charged surface domains. Cy3 works as a donor while the Cy5 functions as a reporter. The second charged surface domain serves as a rotational tool since its charge is orientated with an electric field. The rotational functionality of the FRET adds an extra feature of a conventional FRET system. This rotational functionality allows one to trace conformational changes in regions that have never been examined using crystallographic tools.

Adaptive pharmaceutical. In some embodiments, charged surface domain particles are used to create a new type of cosmetic, pharmaceutical product in a form of a cream, perfume, cosmetic pencil or spray product (e.g., a cosmetic product with biologically active ingredients purporting to have medical or drug-like benefits). For example, in some embodiments, the particles encapsulate in the core one or more of a collagen, anti-aging agent, anti-oxidant, free radical scavenger, moisturizer, depigmentation agent, reflectant, humectant, antimicrobial agent, antibacterial agent, allergy inhibitor, anti-wrinkling agent, antiseptic, analgesic, keratolytic agent, anti-inflammatory inhibitor, low molecular weight molecule, natural macromolecule such as protein or sugar or peptide or DNA or RNA, artificial macromolecules, polymer, dyes, colorant or inorganic ingredient. In these embodiments, the core of these particles release the pharmaceutical agent at different rate (e.g. low or fast) by varying the inherent viscosity of the PLGA polymer or other biodegradable and biocompatible polymers. In some embodiments, a light sensitive agent is incorporated in one charged surface domain while a temperature sensitive agent is incorporated in a second charged surface domain. Changes in light or temperature induce a change in the particle's pharmaceutical effect.

### 2. Materials and Methods for Example Embodiments

2.1 Particles with Single Colored Surface Domains.

The particles depicted in FIG. 3B were formed as described here. Briefly, Poly {D,L-lactide-co-glycolide acid} (PLGA) was dissolved in ethyl acetate. At the same time, PKH26 Red fluorescent cell linker (Sigma, Aldrich of St. Louis, Mo.) was added to 2 milliliters (ml, 1 ml=10⁻³ liters) DSPE-PEG-NH₂ (1 milligram/ml, where 1 milligram, mg,=10⁻³ grams) which was suspended in 4% ethanol. This colored pegylated lipid functional group was sonicated with a thin homogeneizer tip (outer diameter of 10.2 millimeters, mm, where 1 mm=10⁻³ meters; inner diameter of 7.8 mm; length of 113 mm) for 1 minute at 1500 revolutions per minute (rpm). Immediately, PLGA was added to the colored pegylated lipid functional group. The thin homogeneizer tip was located right at the interface formed by ethanol and ethyl acetate. The mixture was sonicated for 1 minute at 4000 rpm. Fifty ml of H₂O was added to each sample. Samples were stirred overnight. Remaining solvent was filtered out using 100 kiloDalton (kDa, 1 kDa=10³ Daltons, 1 Dalton is the mass of a proton) amicon filters.

The particles depicted in FIG. 3D were formed as described here. Briefly, PLGA was dissolved in ethyl acetate. At the same time, BODIPY™ 630/650-X, SE from Invitrogen of Carlsbad, Calif., previously dissolved in 10 microliters (μl, 1 μl=10⁻⁶ liters) of DMF was added to 2 ml of DSPE-PEG-MAL (1 mg/ml). This colored pegylated lipid functional group was sonicated with a thin homogeneizer tip for 1 minute at 1500 rpm. Immediately, PLGA was added to the colored pegylated lipid functional group. The homogeneizer tip was located right at the interface formed by ethanol and ethyl acetate. The mixture was sonicated for 1 minute at 4000 rpm. Fifty ml of H₂0 was added to each sample. Samples were stirred overnight. Remaining solvent was filtered out using 100 kDa amicon filters.

2.2 Microparticle Synthesis with Charged and Colored Multiple Domains

Polymer microparticles were prepared by functionalizing PLGA with 50% DSPE-PEG-NH₂ and 50% DSPE-PEG-MAL using the emulsion method. Briefly, PLGA was dissolved in ethyl acetate. 6-(((4,4-difluoro-5-(2-thienyl)-4-bora-3a,4a-diaza-s-indacene-yl)styryloxy)acetyl)aminohexanoic acid, succinimidyl ester (BODIPY™ 630/650-X, SE from Invitrogen of Carlsbad, Calif.) previously dissolved in 10 μl of DMF was added to 1 ml of DSPE-PEG-MAL (1 mg/ml). At the same time, PKH26 Red Fluorescent Cell Linker (Sigma, Aldrich of St. Louis, Mo.) was also added to 1 ml DSPE-PEG-NH₂ (1 mg/ml). These colored pegylated lipids with functional groups were suspended in 4% ethanol, then mixed and sonicated with a thick homogenizer tip (outer diameter 16.1 mm; inner diameter 12.9 mm; length 165 mm) for 1 minute at 1500 rpm. Immediately, the PLGA was added to the lipid mixture. The homogeneizer tip was located right at the interface formed by ethanol and ethyl acetate. The mixture was sonicated for 1 minute at 4000 rpm. Fifty ml of H₂0 was added to each sample. Samples were stirred overnight. Remaining solvent was filtered out using 100 kDa amicon filters. These samples were characterized with scanning electron and fluorescent microscopes.

2.3 Nanoparticle Synthesis with Dual Charged Color Domains

Poly (D,L-lactide-co-glycolide)-Lipid poly(ethylene glycol) nanoparticles (PLGA-Lipid-PEG NPs) were prepared as follows. PLGA polymer was dissolved in acetonitrile at a concentration of 2 mg/mL. BODIPY™ 630/650 previously dissolved in 10 μl of DMF was added to 120 μl of DSPE-PEG-MAL. At the same time, red fluorescent dye was added to 120 μl of DSPE-PEG-NH₂. These pegylated color lipids with functional groups were mixed with Lecithin (Alfa Aesar) at a molar ratio of 8.5:1.5 and dissolved in 4% ethanol aqueous solution. The total lipid weight (lecithin+DSPE-PEG-COOH) was 15% of the PLGA polymer. The lipid solution was preheated at 65° C. for 3 minutes, and the PLGA solution was added dropwise under gentle stirring. The mixed solution was vortexed vigorously for 3 minutes followed by gentle stirring for 2 hours at room temperature. Finally the nanoparticles were washed three times using an Amicon Ultra-4 centrifugal filter (Millipore, Billerica, Mass.) with a molecular weight cutoff of 10 kDa. The same procedures were used to prepare the hybrid nanoparticles with other surface functional groups or a mixture of surface groups, as enumerated in more detail below.

### 2.4 Electrophoretic Mobility of the Charged Particle

In order to test the electrophoretic mobility of new charged particles, the particles were loaded and run through a native gradient gel (4-12%) at 200 V for several minutes. Non-denaturing buffer was used to run these samples.

### 2.5 Particle Synthesis for Adaptive Pharmaceutical Camouflage

Dual charged surface polymer domain microparticles that can be used as a medicinal cosmetic adaptive camouflage product were prepared by encapsulating collagen or indolepropiomamide in the core of PLGA microparticles. This polymer was dissolved in ethyl acetate. At the same time, PKH26 Red Fluorescent Cell Linker (Sigma, Aldrich) was added to 1 ml DSPE-PEG-NH2 (1 mg/ml) and mixed with a light sensitive polymer. Blue fluorescent dye (BODIPY® 630/650-X, SE) (Invitrogen) was added to 1 ml DSPE-PEG-MAL (1 mg/ml) and mixed with a temperature sensitive polymer. These pegylated lipids with functional groups were suspended in 4% ethanol, then mixed and sonicated with a thick homogenizer tip for 1 min at 1500 rpm. Immediately, the dissolved PLGA was added to the lipid mixture. The homogeneizer tip was located right at the interface formed by ethanol and ethyl acetate. The mixture was sonicated for 1 min at 4000 rpm. Fifty ml of H₂0 was added to each sample. Samples were stirred overnight. Remaining solvent was filtered out using 100 kDa amicon filters. In other embodiments, other anti-aging active ingredients, such as melatonin, are encapsulated in the core of the polymer.

### 3. Color Changes

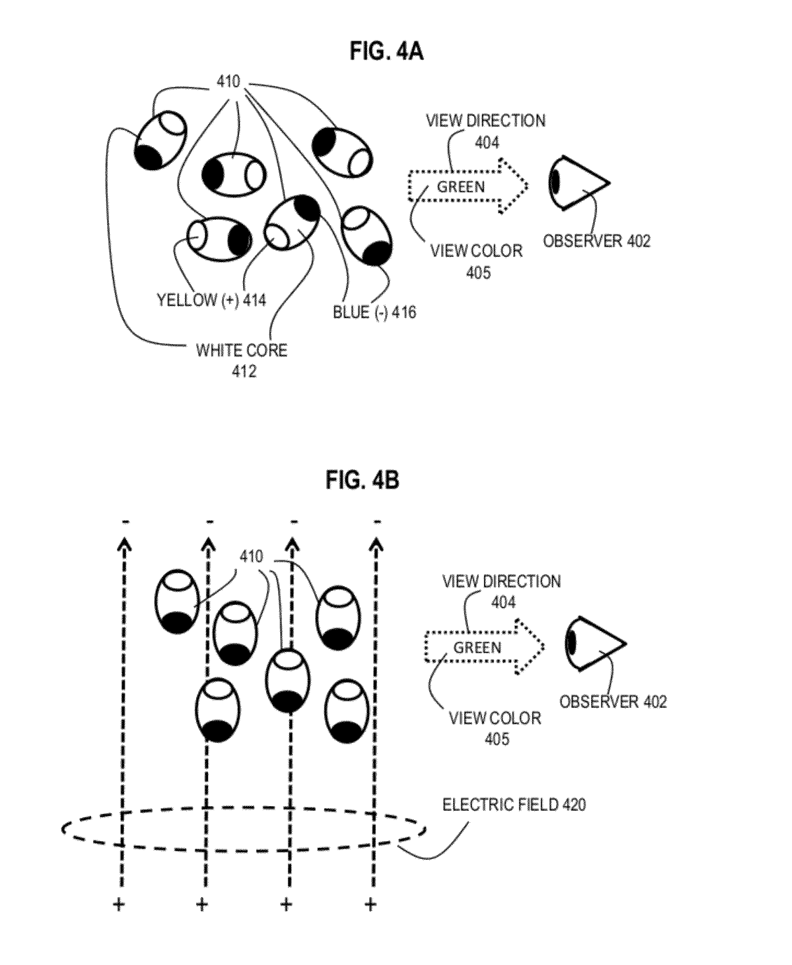

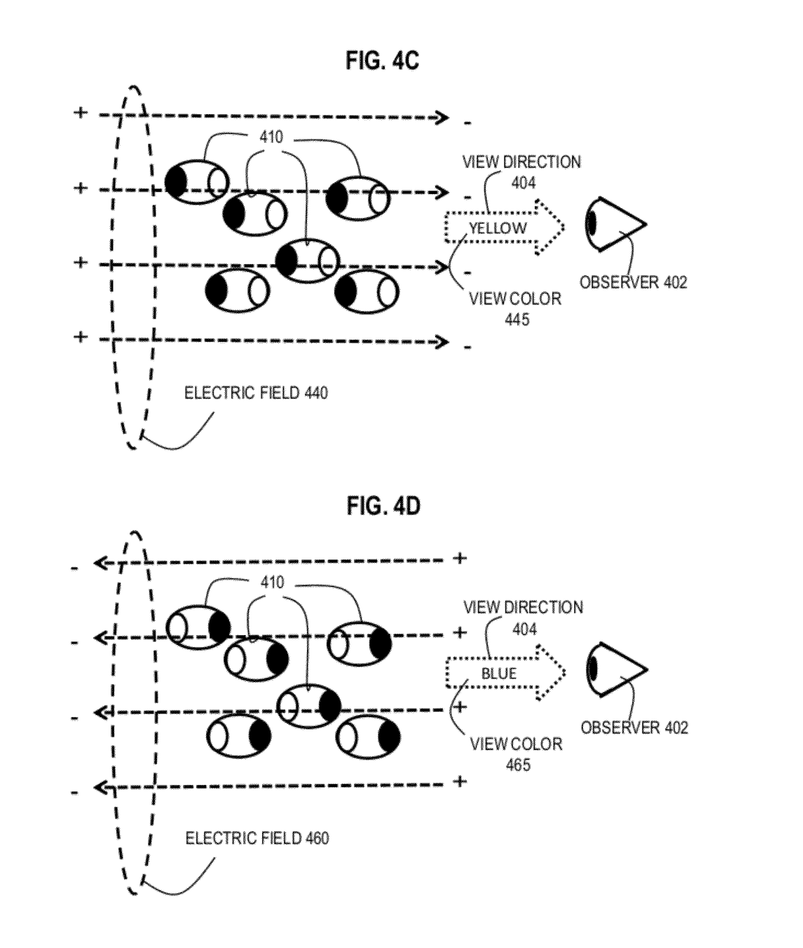

FIG. 4A is a block diagram that illustrates an example color observed from a population of randomly oriented particles that each include two surface domains of different net charge and different dyes, according to an embodiment. In the illustrated example, the core structure 412 is uncolored (white). One surface domain 414 includes a yellow color and a positive charge in the functional group. The other surface domain 416 includes a blue color and a negative charge in the functional group.

When randomly oriented and viewed in a view direction 404 by an observer 402, the population of particles appears with a view color 405 of green, since both yellow and blue surface domains face the observer 402. Because the particles are randomly oriented, the population appears green when viewed in any direction.

FIG. 4B is a block diagram that illustrates an example color observed from a population of particles 410 of FIG. 4A in a first electric field, according to an embodiment. The electric field 420, depicted as arrows oriented from a positive electrode to a negative electrode, is perpendicular to the view direction 404 to the observer 402. If the particles have sufficient mobility, or the field 420 is strong enough, even neutral particles will align so that the positive yellow surface domain is facing the negative electrode and the negative blue surface domain is facing the positive electrode. Viewed in view direction 404 from the side perpendicular to the electric field, both yellow and blue surface domains will be in view of observer 402 and the view color 405 of the population will appear green, as with the random orientations. However, unlike the random orientations, if viewed from above or below, the population will appear predominantly yellow or blue, respectively.

FIG. 4C is a block diagram that illustrates an example color observed from a population of particles 410 of FIG. 4A in a second electric field perpendicular to the first electric field, according to an embodiment. The electric field 440, depicted as arrows oriented from a positive electrode to a negative electrode, is in the view direction 404 to the observer 402. The view color 445 of the population will appear yellow. This is accomplished by the alignment of the electric field and not the movement of the observer 402. Therefore, to the observer 402 the view color of the population appears to have changed from green 405 to yellow 445.

FIG. 4D is a block diagram that illustrates an example color observed from a population of particles 410 of FIG. 4A in a third electric field opposite to the second electric field, according to an embodiment. The electric field 460, depicted as arrows oriented from a positive electrode to a negative electrode, is opposite the view direction 404 to the observer 402. The view color 465 of the population will appear blue. Again, this is accomplished by the alignment of the electric field and not by the movement of the observer 402. Therefore, to the observer 402 the view color of the population appears to have changed from green 405 to yellow 445 to blue 465. As depicted in FIG. 4B through FIG. 4D, the color of the population of particles in the presence of an electric field are changed by changing the polarity of the electric field from positive to negative and vice versa.

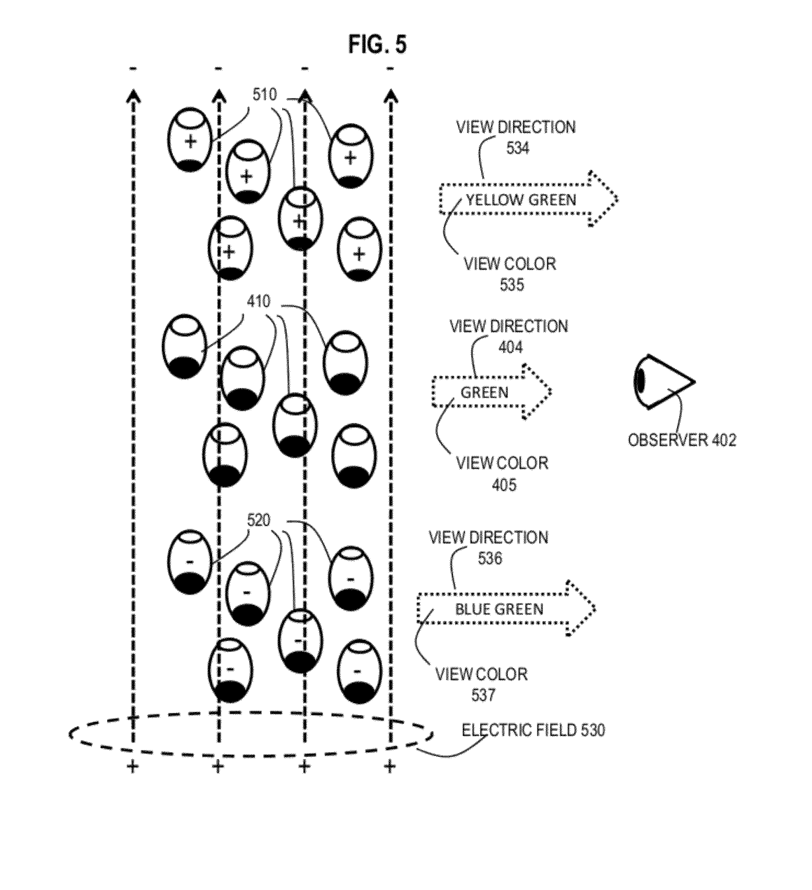

FIG. 5 is a block diagram that illustrates an example range of colors observed in an electric field from a population of particles that each includes two different surface domains but of different total charges, according to an embodiment. This population includes not only the neutral particles 410 of FIG. 4A but also particles 510 and 520. Particles 510 have an excess positive yellow surface domain compared to the negative blue surface domain, so that the particles 510 have a net positive charge. Particles 520 have an excess negative blue surface domain compared to the positive yellow surface domain, so that the particles 520 have a net negative charge. If the particles are sufficiently mobile or the electric field 530 is sufficiently strong, the negative particles 520 migrate toward the positive electrode and the positive particles 510 migrate toward the negative electrode. After some time, the total population will stratify into different sub-populations in different portions of the electric field. To an observer 402 in view direction 404 from the particles 410, the view color is 405 is green, as in FIG. 4B. However, in view direction 534 from the particles 510, the view color 535 is more yellow, called yellow green herein. Similarly, in view direction 536 from the particles 520, the view color 537 is bluer, called blue green herein. Thus, in some embodiments, mixed populations of particles are used to change colors in the presence of a changing electric field.

As another example of mixed populations, in some embodiments, a first population comprising particles with a negative charge and a first color, e.g., a core structure with a first color and a surface domain with a negative charge, is mixed with one or more other populations of particles. For example, one other population of particle has a core structure with a different color and a surface domain with a different charge, such as a positive charge or a more negative charge or a less negative charge. The colors become separated in a steady electric field, and mixed in an alternating electric field. Thus an observer will see the combined color (e.g., green) or the separate colors (e.g., blue and yellow) based on the strength and persistence of the electric fields.

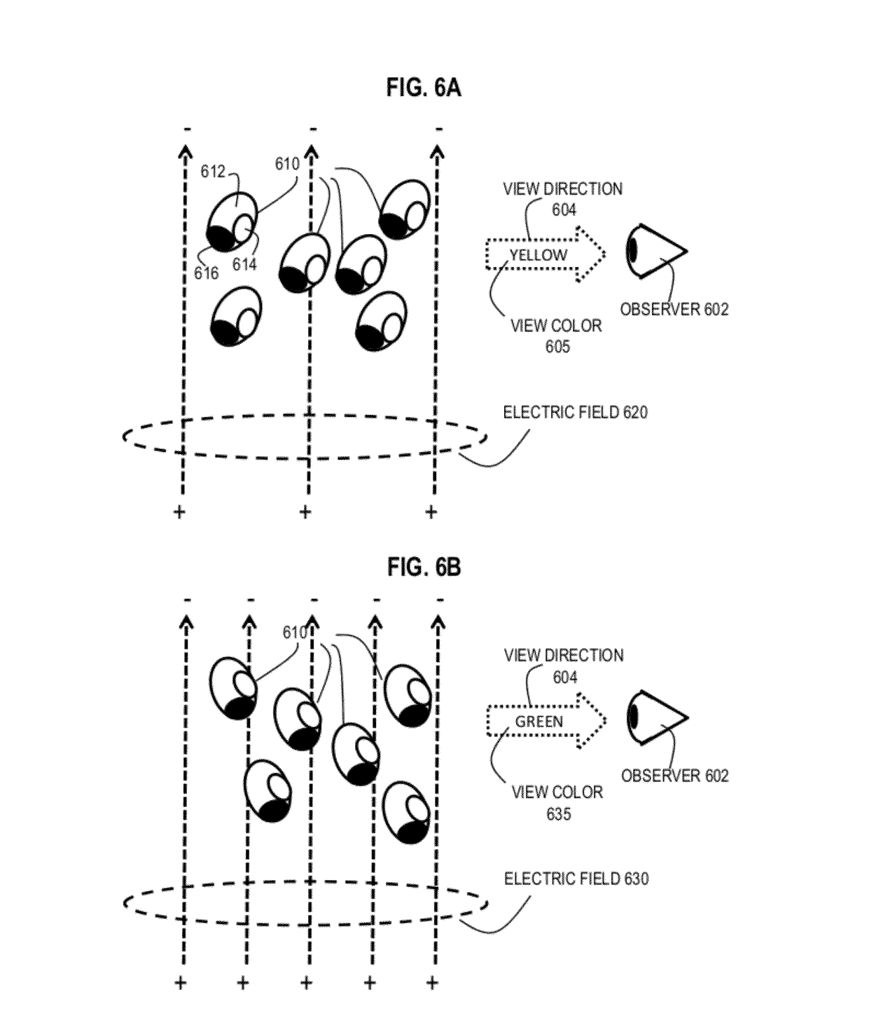

In some embodiments, a reversal of the electric field is not needed, but a change in field strength is sufficient to change the color of the particles. FIG. 6A is a block diagram that illustrates an example color observed from a population of particles 610 that each include two different surface domains in an electric field 620 of a first strength. The particles 610 include a non-symmetric mosaic of surface domains, such as a white core 612 with a positive yellow surface domain 614 and an adjacent negative blue surface domain 616. In the electric field 620 of low strength, or with particles of low mobility, the particles align somewhat with the field 620; but do not achieve a complete alignment. In view direction 604 to observer 602 the blue surface domains are not apparent; and the view color 605 is yellow.

FIG. 6B is a block diagram that illustrates an example color observed from the population of particles 620 of FIG. 6A in an electric field 630 of a different second strength. In the electric field 630 of high strength (e.g., indicated by extra field lines), the particles align more completely with the field 630. In view direction 604 to observer 602 the blue surface domains are apparent; and the view color 635 is green. As depicted in FIG. 6A through FIG. 6B, the color of the population of particles in the presence of an electric field are changed by changing the magnitude voltage. For example, a green ink is displayed when the voltage is 100 mV, and a yellow ink is displayed when the voltage is 75 mV. Similar results may be obtained with other colors.

### 4. Reversible Tattoos

In some embodiments, a single-color tattoo is created with a color electric ink particle made of charged, biodegradable and biocompatible particles, by encapsulating the dye in the core of the particles. In some embodiments, two reversible color tattoos are created with charged polymer particles with two surface domains, by encapsulating one dye in the core and placing the other dye on one of the charged surface domains, or simply by coloring the charged surface domains with two different dyes. By changing the polarity of the electric field and depending on the charge of the surface domains, the desired color will be displayed. This can be repeated at will many times.

In some embodiments, tattoos with three reversible color are created with polymer particles with three functionalized surface domains, by coloring the positive charged surface domain with two different dyes and by coloring the negative charged surface domain with a third dye. By increasing the magnitude of the voltage, the color surface domain that is more positive charged will be displayed when voltage of a specific magnitude is applied. The same rationale is used for the second positive color surface domain. The third color negative surface domain will be displayed when the polarity of the electric field is changed. This can be repeated at will many times.

In some embodiments, tattoos with three super-imposed reversible colors are created with single charged particles containing a dye in their core. The electronic ink is formed by mixing two sets of particles of different charge and color. The color of the tattoo will be the one that emerges from the combination of the particles of different colors. By applying an electric field and changing the polarity of the electric field, the desired color will correlate with the charge of the particle. For example, mixing yellow positive charged particles with blue negative charged particles give rise to green charged particles, and by changing the polarity of the electric field one can produce yellow or blue tattoos.

For example tattoo embodiments, chicken skins were used as a skin model. Before applying the colored charged surface domain particles into the skin, the chicken's leg was cleaned with MQ water to remove any large impurities. A tattoo was made on the chicken skin using a tattoo machine and one of the charged surface domain ink particles. A tissue was passed over the skin in order to determine that the ink did not come off the chicken leg by wiping.

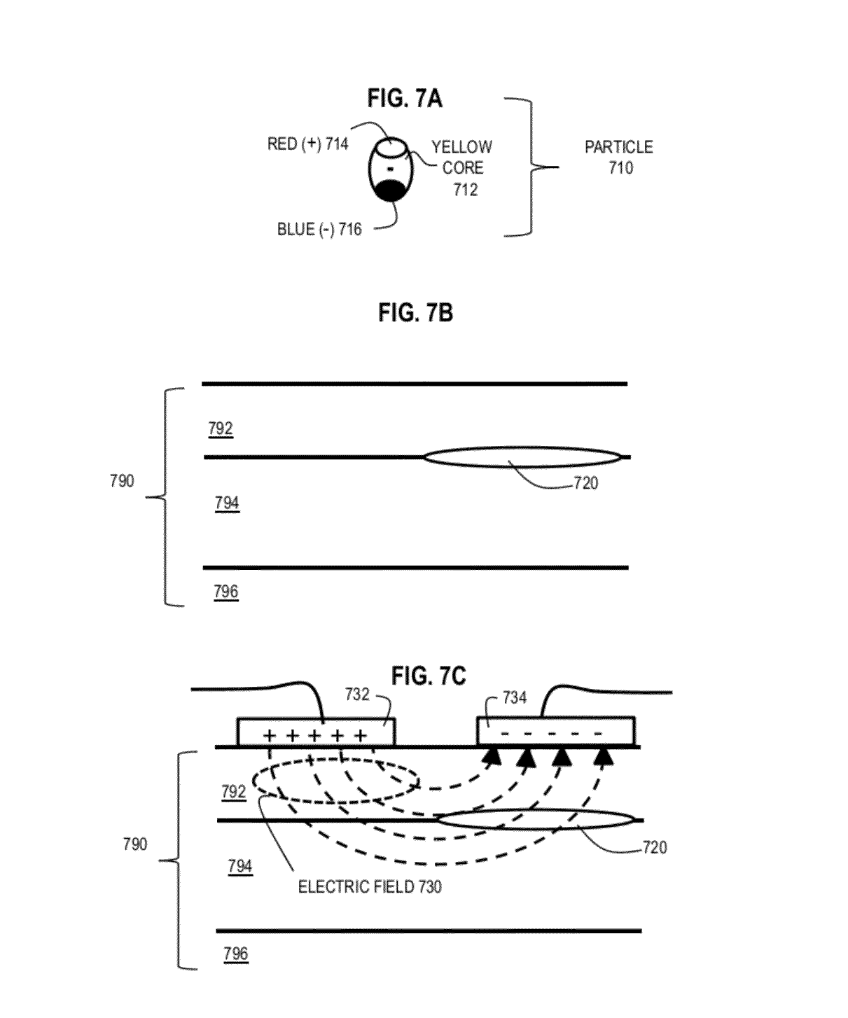

FIG. 7A is a block diagram that illustrates an example particle 710 that includes two surface domains 714 and 716 of different net charges and different dyes and a core structure 712 that includes a third dye, according to an embodiment. In this illustrated embodiment, the core structure 712 includes a yellow dye, one surface domain includes a negatively charged blue dye functional group and the other surface domain includes a positively charged red dye functional group. A population of particles 710 was produced by functionalizing a core labeled with NBD dye, a yellow green color, with 50% DSPE-PEG-MAL linkers and 50% DSPE-PEG-NH₂ linkers. The first linker was labeled with blue fluorescent dye(−) while the latter linker was colored with red fluorescent dye(+). The overall charge of these particles is negative because of a negative charge contribution from the core structure 712. The functionalized surface domains do not completely cover the particle's surface; therefore the charge of the core contributes to the global charge of the particle.

Although surface domains 714 and 716 are shown on opposite sides of particle 710 for purposes of illustration, in various embodiments, the population includes particles with differently arranged surface domains, of varying relative positions, sizes and total charges. In some embodiments, the population includes some particles with a single surface domain and some particles with no surface domains.

These particles are safe for use in humans because the particles' compositional components are biocompatible. The core is made of a biodegradable and biocompatible polymer (PLGA) that is already approved by the FDA. The charged surface domains are formed by pegylated lipid functional groups which are also biocompatible. For example, PLGA particles functionalized with 100% DSPE-PEG-NH₂, 100% DSPE-PEG-COOH, and 100% DSPE-PEG-OCH₃ were reported to be blood compatible (Salvador-Morales, C.; Zhang, L.; Langer, R.; Farokhzad, O. C. “Immunocompatibility properties of lipid-polymer hybrid nanoparticles with heterogeneous surface functional groups,” Biomaterials v. 30 pp 2231-2240, 2009). According to that paper, in several measured combinations, such particles activate the complement system in blood to a lesser extent than does Zymosan, a well-known activator of the complement system. The complement system is part of the immune system and is the most important system in the blood for recognition of foreign materials. To date, it is known that the activation of the complement system is mainly due to the surface chemistry of the charged surface domains microparticles. Because the surface chemistry of these particles induces low levels of complement system activation in blood, it is unlikely that these microparticles cause any major adverse reaction on skin such as inflammation, keloids (scars that grow beyond normal boundaries) or formation of granulomas. The formation of granulomas, as well as inflammation, is closely related to the activation of the complement system not only in the blood stream but also in other tissues including the skin. Therefore, the surface chemistry of these particles is not expected to cause a harmful effect on the skin. Nevertheless, other toxicity assays in vitro and in vivo are underway in order to assess the toxicity profiles of the other elements involved in this electronic ink—such as the use of dyes and pigment.

To date, the toxicity profile of tattoos is closely related to the chemical composition of the tattoo ink. This is because the tattoo ink in most of the cases is made of pigments which are completely and permanently exposed to the skin. In some embodiments described herein, the dyes or pigments are encapsulated in the core of the microparticle and the rate of biodegradability of such particles is extremely low, as described below. Thus the particles in such embodiments represent an advantage because such particles display colors while the contact with the skin is minimal. The toxicity of the electronic ink particles for tattoos is related to the nature of the pigments and not to the composition of the polymer carriers. The versatile method of synthesis allows the encapsulation of a wide range of dyes and pigments. Thus it is advantageous to select pigments or dyes that are minimally harmful. In some embodiments with dyes in the charged surface domains, the dye is exposed to the skin; however, in temporary tattoos that are removed by electric field, the exposure can be controlled. In addition, in some embodiments, the colored particles are applied to the epidermis layer of the skin; and, not to the hypodermis layer. This reduces the migration of the pigments to the nodes of the adjacent tissues.

FIG. 7B is a block diagram that illustrates an example tattoo comprising a population 720 of the particles 710 of FIG. 7A, according to an embodiment. The population 720 is deposited in the skin 790 with a tattoo machine. The skin 790 includes an epidermis layer 792, a dermis layer 794 and a hypodermis layer 796. For purposes of illustration, the population is shown between the epidermis layer 792 and dermis layer 794 of the skin 790, but in other embodiments, the population of ink particles is distributed in a different location in the skin 790. After insertion, the population 720 of ink particles in the tattoo is evident as a yellow brown area as a result of the contributions from the red, blue and yellow dyes in various portions of the randomly oriented particles 710.

FIG. 7C is a block diagram that illustrates an example tattoo comprising a population 720 of the particles 710 of FIG. 7A in the presence of an electric field 730, according to an embodiment. The population 720 and skin 790 are as described above. The electric field 730 is illustrated as field lines extending from a positively charged electrode 732 to a negatively charged electrode 734. If the particles 710 are mobile enough or the field 730 is strong enough, the particles in population 720 will orient, at least to some extent, with the electric field. Because the skin 790 is conductive, a current will flow between the electrodes 732 and 734. In the depicted electric field, the particles will orient with the positive red surface domains directed to the surface, and the tattoo will appear more red. In a reversed electric field, the particles will orient with the negative blue surface domains directed to the surface, and the tattoo will appear more blue. In an even stronger electric field 730, if the particles are mobile enough, the negatively charged particles 710 in population 720 will migrate toward the positively charged electrode 732.

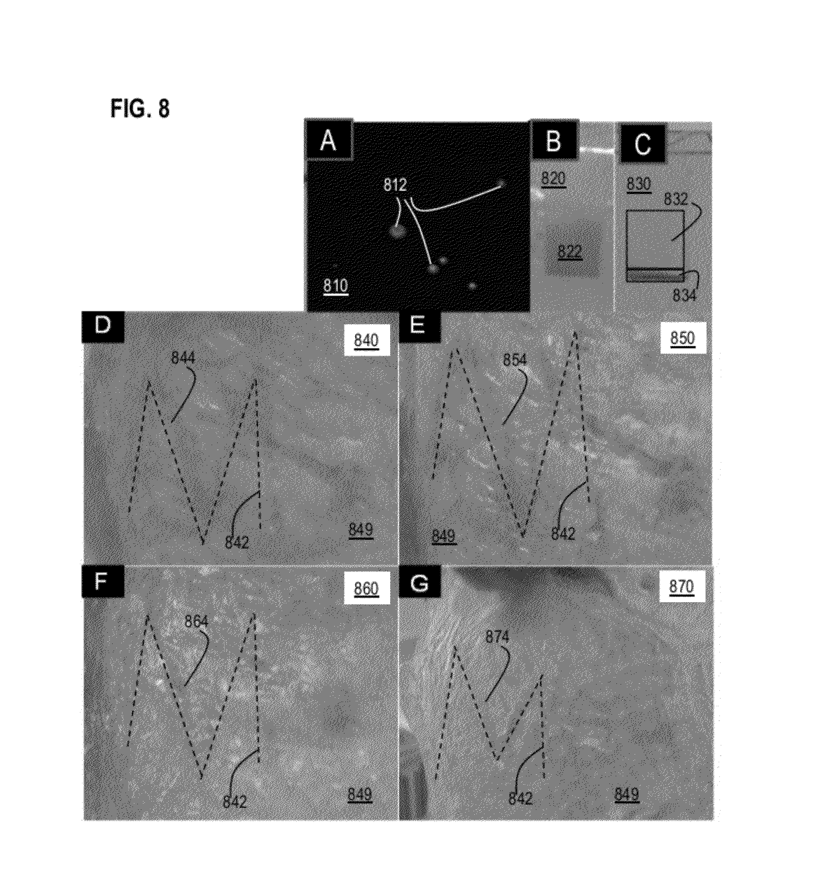

FIGS. 8A through 8G are photographs that illustrates colors observed from a population of the particles 710 of FIG. 7A before and after applying an electric field, according to various embodiments. FIG. 8A is a fluorescent micrograph of microparticles synthesized as described above for particle 710. The microparticles 812 fluoresce green due to the NBD dye in the core structure 712. Red and blue fluorescence were also observed due to the dyes in the two surface domains. FIG. 8B is a photograph 820 of the population of particles 822 deposited in a gel. In this configuration the population appears a brownish yellow due to contributions from all three colors of the randomly oriented particles 710.

FIG. 8C is a photograph 830 that shows the dye after being driven through the gel by an electric field of 200 volts upward for five minutes. The particles with predominant positively charged red surface domains appear in regions 832, while the negatively charged predominately yellow and blue particles appear in region 834.

FIG. 8D is a photograph 840 of a chicken skin 849 with a M-shaped tattoo 844 just outside the dashed M shape 842. The tattoo comprises the population 720 of particles 710 before orientation in an electric field, and appears a dark brownish yellow as in photograph 820 of FIG. 8B.

FIG. 8E is a photograph 850 of the chicken skin 849 with the M-shaped tattoo 854 just outside the dashed M shape 842, after placing two adhesive electrodes and passing a current of 25 milliAmperes (mA, 1 mA=10⁻³ Amperes) for five minutes. The tattoo color 854 is becoming slightly reddish as the skin 849 lightens during drying. FIG. 8F is a photograph 860 of the chicken skin 849 with the M-shaped tattoo 864 just outside the dashed M shape 842 after again applying 25 mA for five minutes. The color 864 is more reddish. FIG. 8G is a photograph 870 of the chicken skin 849 with the M-shaped tattoo 874 just outside the dashed M shape 842 in which the color 874 is reddish. This demonstrates that by applying an electric field the color of the tattoo is changed; in this embodiment from yellow to red.

In various other embodiments, other populations of particles are used as tattoo inks For example, in some embodiments, microparticle surfaces were functionalized with 100% DSPE-PEG-NH₂(+) and blue fluorescent dye was encapsulated in particle's core. These particles have a positive charge. In some embodiments, microparticle surfaces were functionalized with 100% DSPE-PEG-MAL; and NBD dye was encapsulated in particle's core. These particles have a negative charge and appear green. In some embodiments, microparticle surfaces were functionalized with 100% DSPE-PEG-NH₂; and a red fluorescent dye was encapsulated in the particle's core. These particles have a positive charge. In some embodiments, microparticle surfaces were functionalized with 100% DSPE-PEG-MAL(−); and NBD dye (yellow-green color) was encapsulated in the particle's core. In some embodiments, microparticle surfaces were functionalized with 50% DSPE-PEG-MAL(−) and 50% DSPE-PEG-NH₂(+). The 50% DSPE-PEG-NH₂ surface domain was labeled with red fluorescent dye while NBD dye was encapsulated in the particle's core. The overall charge of these particles is negative because of the core's charge contribution. In some embodiments microparticle surfaces were functionalized with 50% DSPE-PEG-MAL(−) and 50% DSPE-PEG-NH₂(+). The first linker was labeled with blue fluorescent dye(−) while the latter was colored with red fluorescent dye(+). The core was not labeled but had a net negative charge. The overall charge of these particles is negative because of the core's charge contribution. The functionalized surface domains do not completely cover the particle's surface; therefore the charge of the core contributes to the global charge of the particle.

The electrophoretic mobility of these particles depends on the particle's surface domains and/or color. Nine populations of particles were synthesized and subjected to electrophoretic mobility measurements. The nine particle populations include the following. 1. PLGA-50% DSPE-PEG-NH₂-Red fluorescent dye/50% DSPE-PEG-MAL. 2. PLGA-50% DSPE-PEG-NH₂-Red fluorescent/50% DSPE-PEG-MAL+PLGA-50% DSPE-PEG-MAL-Blue fluorescent/50% DSPE-PEG-NH₂-red fluorescent. 3. PLGA-50% DSPE-PEG-MAL-Blue fluorescent dye+PLGA-50% DSPE-PEG-NH₂. 4. PLGA-50% DSPE-PEG-MAL-Blue fluorescent dye+50% PLGA-DSPE-PEG-NH₂. 6. PLGA-50% DSPE-PEG-NH₂-red fluorescent-dye+PLGA-50% DSPE-PEG-MAL-Blue fluorescent dye+Core-NBD. 7. PLGA-50% DSPE-PEG-NH₂-red fluorescent-dye+PLGA-50% DSPE-PEG-MAL-Blue fluorescent dye+Core-NBD. 9. PLGA-50% DSPE-PEG-NH₂-red fluorescent-dye+PLGA-50% DSPE-PEG-MAL-Blue fluorescent dye+unlabeled core. The samples were run at 200V for 3 minutes and the polarity of the electric field was changed several times from positive to negative and vice versa. Particles whose two functionalized surface domains are colored with red and blue and that also have a yellow core display three colors: red (top), blue (bottom), yellow (bottom). The yellow core dominates over blue so the blue color is often not visible. Particles that have two colored surface domains (e.g. red and blue) display blue on top and red on bottom. When the polarity changed the colors inverted themselves. The separation of the two colors was clearly observed.

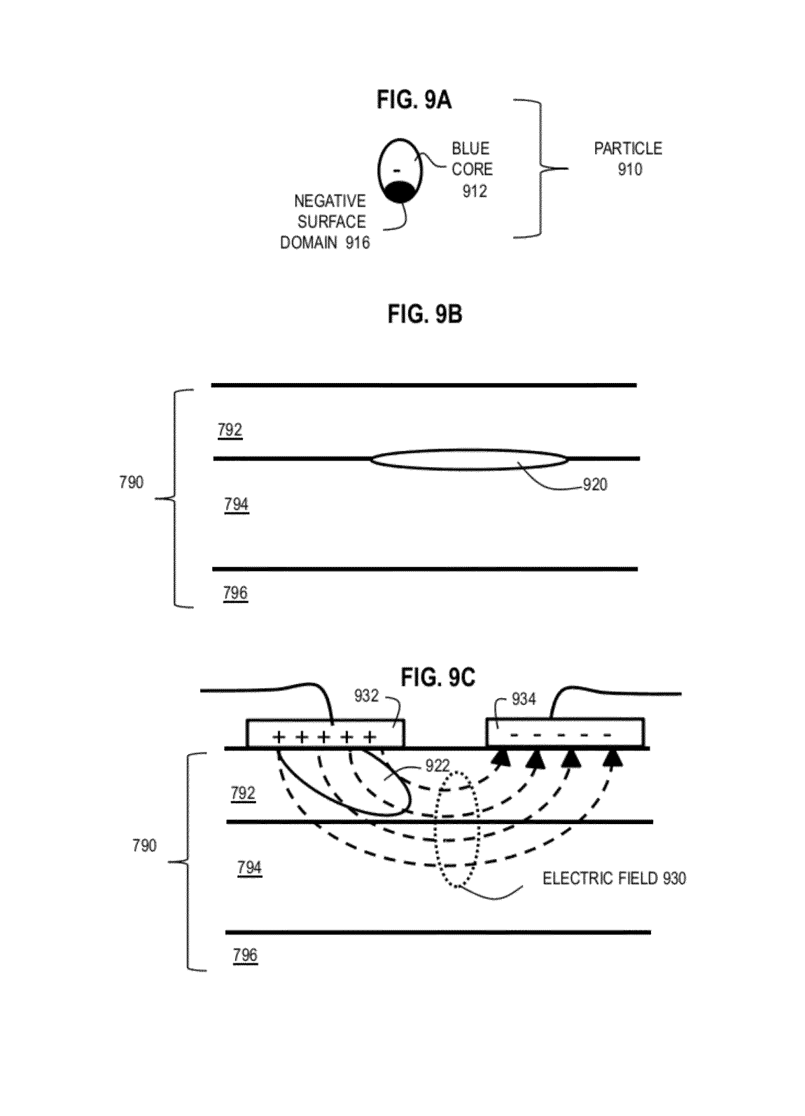

In some embodiments, the electrophoretic mobility of the particles is used to remove them from skin and hence provide removable tattoos. FIG. 9A is a block diagram that illustrates an example particle 910 that includes a single surface domain 916 of negative net charge and blue dye, according to an embodiment. The particle's surface was functionalized with DSPE-PEG-MAL(−) and a blue fluorescent dye was encapsulated in particle's core structure 912. The overall charge of this particle is negative.

FIG. 9B is a block diagram that illustrates an example tattoo comprising a population 920 of the particles 910 of FIG. 9A, according to an embodiment. The skin 790 is as described above for FIG. 7B. The tattoo appears blue when viewed from above the surface of the skin 790.

FIG. 9C is a block diagram that illustrates an example tattoo comprising a population 922 of the particles 910 of FIG. 9A after application of an electric field 930, according to an embodiment. The skin 790 is as described above for FIG. 7B. The electric field 930 is illustrated as field lines extending from a positively charged electrode 932 to a negatively charged electrode 934. Because the skin 790 is conductive, a current will flow between the electrodes 932 and 934. If the electric field 930 is strong enough, and if the particles 910 are mobile enough, the negatively charged particles 910 in population 920 will migrate toward the positively charged electrode 932 to become the migrated population 922 adjacent to the positive electrode 932. If the electric field is reversed repeatedly, the population of particles will migrate to both electrodes (not shown). At the surface of the skin 790, the migrating negatively charged particles 710 will contribute to the current flow and become affixed to the positive electrode 932.

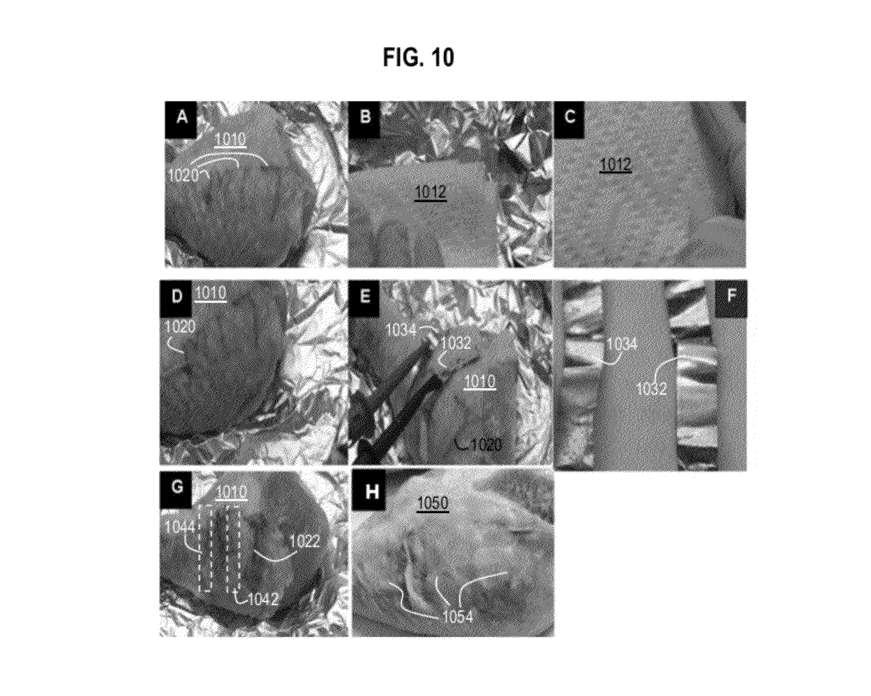

FIGS. 10A through 10H are photographs that illustrate example migration of the particles of FIG. 9A after applying an electric field, according to various embodiments. FIG. 10A is a photograph of chicken skin 1010 with a tattoo 1020 comprising a population of the negatively charged blue particles 910. The tattoo spells the letters “MIT”. FIG. 10B is a photograph depicting the chicken skin being wiped by a paper towel 1012. FIG. 10C is a photograph depicting the wiping surface of the paper towel 1012 without a blue mark. FIG. 10D is a photograph that depicts the chicken skin 1010 and tattoo 1020 after wiping. Thus FIGS. 10B through 10D demonstrate that the population of particles 910 formed a tattoo in the skin and is not easily removed from the surface.

FIG. 10E is a photograph that depicts a positive electrode 1034 and a negative electrode 1032 placed on the surface of the chicken skin 1010. Electrodes 1032 and 1034 were placed on the chicken skin 1010 in order to pass a current. Positive electrode 1034 is over the left edge of the letter “M” of the tattoo 1020; and, the negative electrode 1032 is over the letter “I” of the tattoo 1020. FIG. 1OF depicts the electrodes 1032 and 1034 after passing 25 mA of current through the skin 1010. Positive electrode 1034 showed blue particles. No blue particles were found on negative electrode 1032. FIG. 10G depicts the chicken skin 1010 after passing the electric current. The approximate positions of the positive and negative electrodes are depicted as rectangles 1044 and 1042, respectively. Part of the “MIT” tattoo has been removed leaving portion 1022 of the tattoo and collecting blue particles in the skin between the approximate locations 1042 and 1044 of the negative electrode and positive electrode, respectively. The letter “I” of the tattoo 1020 is essentially absent. Thus, it is apparent that the particles have moved away from the position 1042 of the negative electrode and toward the position of the positive electrode 1044.

FIG. 10H is a photograph depicting another tattoo 1054 on a different chicken skin 1050 after being stored in moist conditions for 1½ months. The tattoo 1024 is evident, and demonstrates that the particles are long lived without electric current removal.

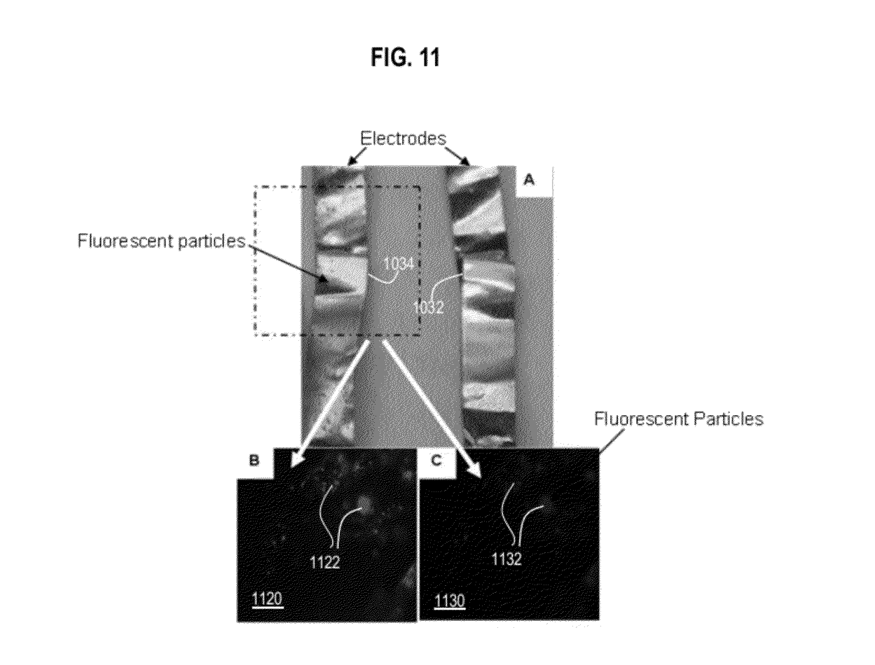

FIGS. 11A through 11C are photographs that illustrate example deposition of the particles 910 of FIG. 9A on a positive electrode, according to an embodiment. FIG. 11A, like FIG. 10F, depicts the electrodes 1032 and 1034 after passing 25 mA of current through the chicken skin 1010. FIG. 11B is a micrograph 1120 showing particles 1122 on the surface of the positive electrode 1032. FIG. 11C is a fluorescent micrograph 1130 showing blue fluorescent particles 1132 that correspond to the particles 1122 in the micrograph 1120. The blue fluorescent microparticles are consistent with the particles 910 used in the tattoo. This corroborates that the electric ink can be removed from a tattoo using an electric field.

In order to improve the efficiency of removing the electronic ink from chicken skin, in some embodiments, professional adhesive electrodes were used. Two dura-stick self-adhesive electrodes were placed on a tattooed chicken leg. A current of 25 mA was applied to the chicken leg using a DC power supply. Electrodes were removed after 5 min. 75% of the mark was removed by this method. The previously used electrodes 1032 and 1034 left many of the particles on the skin surface, forming a blue stained area, due to lack of proper contact. The blue stained areas were not observed using the self adhesive electrodes, and a more clean tattoo removal was achieved.

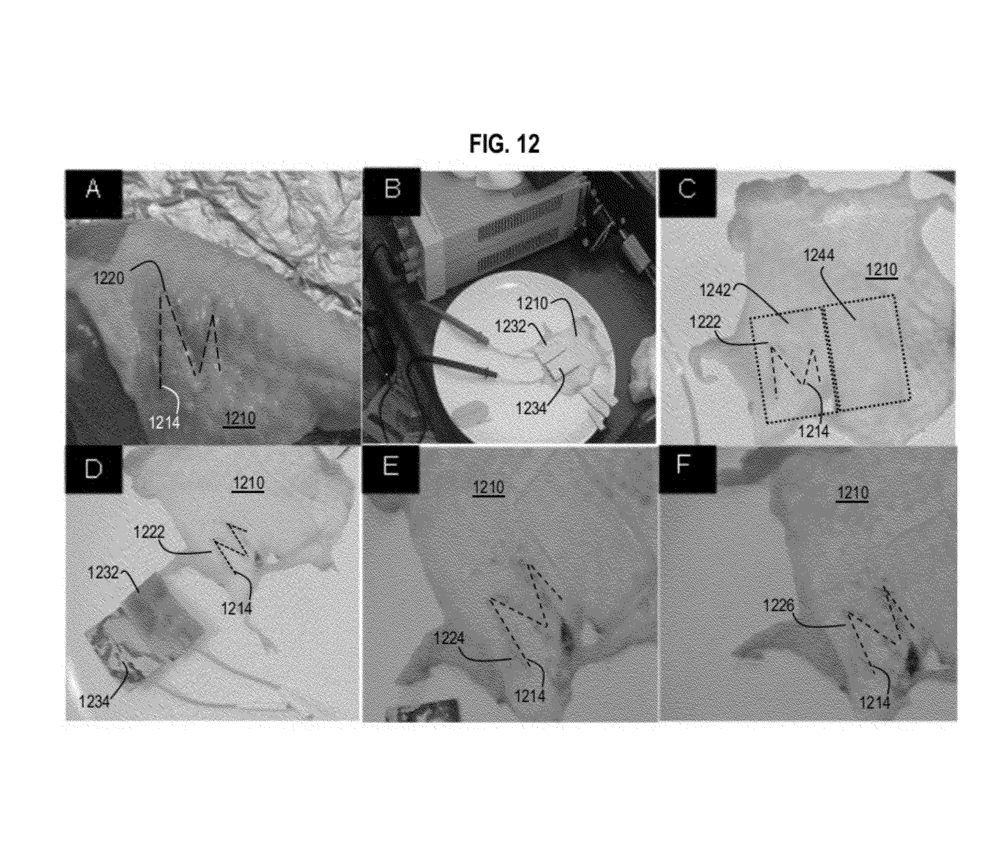

FIGS. 12A through 12F are photographs that illustrate example removal of the particles 910 of FIG. 9A after applying an electric field, according to various embodiments using adhesive electrodes. FIG. 12A is a photograph of chicken skin 1210 with a tattoo 1220 comprising a population of the negatively charged blue particles 910 in the shape of an M just outside the dashed lines 1214.

FIG. 12B depicts the chicken skin 1210 with positively charged adhesive electrode 1232 and negatively charged adhesive electrode 1234 connected to voltage source to drive a 25 mA current for five minutes. FIG. 12C depicts the chicken skin 1210 after passing the electric current. The approximate positions of the positive and negative electrodes are depicted as rectangles 1242 and 1244, respectively. Part of the M tattoo outside the dashed lines 1214 has been removed leaving portion 1222 of the tattoo. FIG. 12D depicts the adhesive electrodes 1232 and 1234 after passing 25 mA of current through the skin 1210. Positive electrode 1232 showed blue particles. No blue particles were found on negative electrode 1234. The chicken skin 1210 and the remaining M shaped tattoo portion 1222 outside the dashed lines 1214 are also shown. FIG. 12E depicts the chicken skin 1210 after passing another electric current. Another portion of the M tattoo outside the dashed lines 1214 has been removed leaving portion 1224 of the tattoo. FIG. 12F depicts the chicken skin 1210 after applying 25 mA three consecutive times for 5 min each time. About 75% of the ink particles were removed from the chicken skin 1210, leaving 25% of the blue particles in the M shaped tattoo portion 1226 outside the dashed lines 1214. This further corroborates that the electric ink can be removed from a tattoo using an electric field.

In still other embodiments, marks were made on a cotton fabric using a tattoo machine and a negative blue ink particle. Adhesive electrodes were placed on the marks and 37 mA were applied for 5 min. The marks were partially removed after applying the current.

### 5. Biodegradable Markings

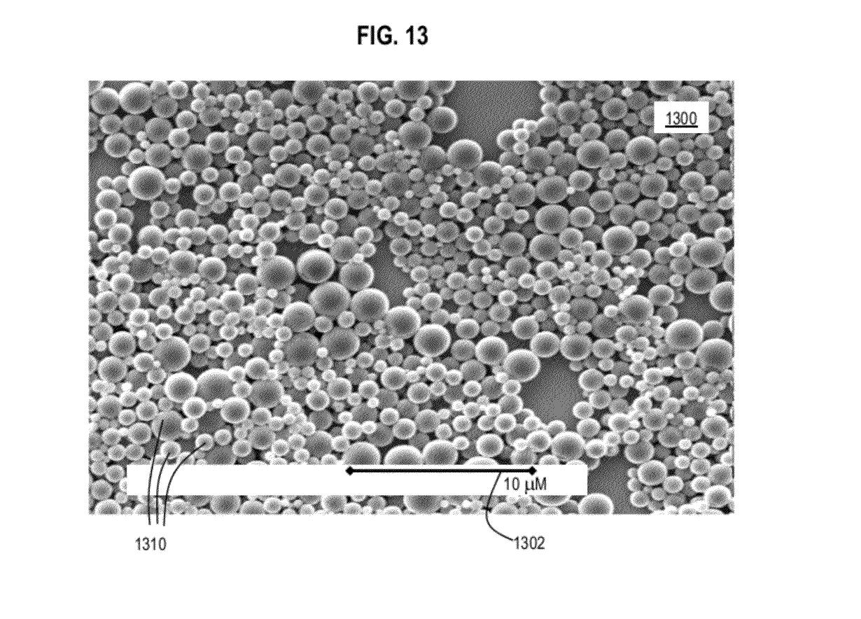

The charged particles can be rather permanent. FIG. 13 is a micrograph 1300 that illustrates example duration of particles 1310 after six months in an aqueous environment indicative of biodegradability, according to an embodiment. The scale 1302 depicts 10 microns. Both microparticles and nanoparticle are included. These microparticles were functionalized with 100% DSPE-PEG-MAL(−). This demonstrates that these particles degrade at an extremely low rate. After being stored for six months in H₂0, the morphology of the particles is intact.

In some embodiments, it is desirable that the particles degrade more rapidly. In such embodiments, the degradation is speeded by modifying the synthesis procedure. For example, By changing the lactide:glycolide ratio and inherent viscosity range of the PLGA polymer, the degradation rate of the polymer can be tuned. It was found that 50:50 lactide:glycolide has one of the fastest degradation rates of the polymer via hydrolysis. Furthermore, nanoparticles are expected to degrade faster than microparticles. In such conditions, tattoos made with nanoparticles with this lactide: glycolide ratio will likely last 1-3 months, thus imparting a subject with a temporary tattoo.

### 6. Other Embodiments

Other particles for reversible tattoos that depend on temperature, light, humidity, conductivity or pH, or some combination, are also used in some embodiments.

Sensitivity to pH and temperature. Dual charged surface polymer domain microparticles sensitive to pH and temperature were prepared by functionalizing a polymer solution mixture composed of poly(N,N-dimethylamino)ethyl methacrylate (DMAEMA)-co-ethyl acrylamide (EAAm) with 100% DSPE-PEG-MAL, 100% DSPE-PEG-NH2, or 50% DSPE-PEG-MAL/50% DSPE-PEG-NH2 using the emulsion method. Briefly, polymer solution mixture composed of poly(N,N-dimethylamino)ethyl methacrylate (DMAEMA)-co-ethyl acrylamide (EAAm) was dissolved in ethyl acetate. This mixture of polymer formed the core of the particle. At the same time, 2 ml of DSPE-PEG-MAL or DSPE-PEG-NH2 was suspended in 4% ethanol, mixed and sonicated with a thick homogenizer tip for 1 minute at 1500 rpm. Immediately, the polymer solution mixture composed of polyDMAEMA, poly EAAm was added to the lipid mixture. The homogeneizer tip was located right at the interface formed by ethanol and ethyl acetate. The mixture was sonicated for 1 minute at 4000 rpm. Fifty ml of H₂O was added to each sample. Samples were stirred overnight. Remaining solvent was filtered out using 100 kDa amicon filter.

Sensitivity to light. Dual charged light sensitive surface domains microparticles were prepared by functionalizing polymer solution mixture composed of poly(N,N-dimethylamino)ethyl methacrylate (DMAEMA)-co-ethyl acrylamide (EAAm) with 100% DSPE-PEG-MAL, 100% DSPE-PEG-NH₂, or 50% DSPE-PEG-MAL/50% DSPE-PEG-NH₂ using the emulsion method. Briefly, polymer solution mixture composed of poly(N,N-dimethylamino)ethyl methacrylate (DMAEMA)-co-ethyl acrylamide (EAAm) was dissolved in ethyl acetate. At the same time, 2 ml of DSPE-PEG-MAL or DSPE-PEG-NH₂ were suspended in 4% ethanol, mixed them and sonicated with a thick homogenizer tip for 1 minute at 1500 rpm. Immediately, the polymer solution mixture composed by polyDMAEMA, poly EAAm was added to the lipid mixture. The homogeneizer tip was located right at the interface formed by ethanol and ethyl acetate. The mixture was sonicated for 1 minute at 4000 rpm. Fifty ml of H₂O was added to each sample. Samples were stirred overnight. Remaining solvent was filtered out using 100 kDa amicon filters. It is important to mention that the light sensitive polymer can be used to form the core of the particles or can serve to form one of the domains. This means that polymers such as poly(2,5-bis(3-sulfonatopropoxy)-1,4-phenylene, disodium salt-alt-1,4-phenylene) can replace the DSPE-PEG-MAL or DSPE-PEG-NH₂. The light sensitive polymer is incorporated in the reaction exactly the same as pegylated lipids are incorporated in the formulation. In other embodiments, other sensitive light polymers are used to form the core of the particles or are encapsulated in the core, such as: poly[(O-phenylenevinylene)-alt-(2-methoxy-5-(2-ethylhexyloxy)-p-phenylenevinylene)], poly[(p-phenylenevinylene)-alt-(2-methoxy-5-(2-ethylhexyloxy)-p-phenylene vinylene)], poly[9,9-bis-(2-ethylhexyl)-94-fluorene-2,7-diyl].

Sensitivity to conductivity. Dual charged surface polymer domain microparticles that are conductive sensitive were prepared forming the core of the particle with poly(3-butylthiophene-2,5-diyl). This polymer was dissolved in ethyl acetate. At the same time, 2 ml of DSPE-PEG-MAL or DSPE-PEG-NH₂ was suspended in 4% ethanol, mixed and sonicated with a thick homogenizer tip for 1 minute at 1500 rpm. Immediately, the polymer solution mixture was added to the lipid mixture. The homogeneizer tip was located right at the interface formed by ethanol and ethyl acetate. The mixture was sonicated for 1 miute at 4000 rpm. Fifty ml of H₂O was added to each sample. Samples were stirred overnight. Remaining solvent was filtered out using 100 kDa amicon filters. In other embodiments, other conductive polymers are used to form the core of the particle or are encapsulated in the particle, including: poly(3-cyclohexyl-4-methylthiophere-2-5-diyl) and poly(9,9-di-(2-ethylhexyl)-9H-fluorene-2,7-vinylene).

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense.

What is claimed is:

1. A method comprising forming a tattoo visible, at least occasionally, on a surface of skin by introducing, into a layer of the skin, a plurality of particles, each particle comprising a core structure and at least one surface domain, wherein the at least one surface domain comprises a net charge and wherein at least one of the core structure or the surface domain comprises a dye.

2. A method as recited in claim 1, further comprising:
placing an electrode near a surface of the skin; and
applying an electric field through the electrode sufficient to cause the plurality of particles to move from the layer of skin toward the electrode.

3. A method as recited in claim 2, further comprising causing at least a portion of the plurality of particles to be deposited on the electrode to remove at least the portion of the plurality of particles from the skin.

4. A method as recited in claim 1, wherein the plurality of particles is biodegradable.

5. A method as recited in claim 1, wherein at least some of the plurality of particles are microparticles with a largest dimension no greater than about 1000 microns.

6. A method as recited in claim 1, wherein at least some of the plurality of particles are nanoparticles with a largest dimension no greater than about 1000 nanometers.

7. A method comprising forming a tattoo visible on a surface of skin by introducing, into a layer of the skin, a plurality of particles, each particle comprising a core structure and a first surface domain and a different second surface domain, wherein:
the first surface domain comprises a first dye and a first net charge,
the second surface domain comprises a different second net charge, and
the first dye is substantively absent in the core structure and in the second surface domain.

8. A method as recited in claim 7, wherein the second surface domain further comprises a different second dye.

9. A method as recited in claim 7, wherein the core structure further comprises a different second dye.

10. A method as recited in claim 7, further comprising:
placing an electrode near a surface of the skin; and
applying an electric field through the electrode sufficient to cause the plurality of particles to orient in the skin based on the electric field.

11. A method as recited in claim 10, wherein applying the electric charge sufficient to cause the plurality of particles to orient further comprises applying the electric charge sufficient to orient the first domain toward the surface of the skin so that the first dye is evident at the surface of the skin.

12. A method as recited in claim 10, wherein applying the electric charge sufficient to cause the plurality of particles to orient further comprises applying the electric charge sufficient to orient the second domain toward to the surface of the skin so that the first dye is not evident at the surface of the skin.

13. A method as recited in claim 12, wherein the second surface domain further comprises a different second dye and the second dye is evident at the surface of the skin.

14. A method as recited in claim 7, wherein the plurality of particles is biodegradable.

15. A method as recited in claim 7, wherein at least some of the plurality of particles are microparticles with a largest dimension no greater than about 1000 microns.

16. A method as recited in claim 7, wherein at least some of the plurality of particles are nanoparticles with a largest dimension no greater than about 1000 nanometers.

17. A particle comprising a core structure and a first surface domain and a different second surface domain, wherein:
the first surface domain comprises a first dye and a first net charge;
the second surface domain comprises a different second net charge; and
the first dye is substantively absent in the core structure and in the second surface domain.

18. A particle as recited in claim 17, wherein the second surface domain further comprises a different second dye.

19. A particle as recited in claim 17, wherein the particle is biodegradable.

20. A particle as recited in claim 17, wherein the particle is a microparticle with a largest dimension no greater than about 1000 microns.

21. A particle as recited in claim 17, wherein the particle is a nanoparticle with a largest dimension no greater than about 1000 nanometers.

22. A particle as recited in claim 17, wherein at least one of the first surface domain or the second surface domain or the core structure further comprises a temperature sensitive agent.

23. A particle as recited in claim 17, wherein at least one of the first surface domain or the second surface domain or the core structure further comprises a pH sensitive agent.

24. A particle as recited in claim 17, wherein at least one of the first surface domain or the second surface domain or the core structure further comprises a light sensitive agent.

25. A particle as recited in claim 17, wherein at least one of the first surface domain or the second surface domain or the core structure further comprises a conductivity sensitive agent.

26. A particle having multiple functionalized surface domains, the particle comprising:
a core structure having a surface;
a plurality of first linkers, each comprising a first end that binds to the surface of the core structure, and a second end that comprises a first functional group having a first charge; and
a plurality of second linkers, each comprising a first end that binds to the surface of the core structure, and a second end that comprises a second functional group having a different second charge;
wherein
the linkers are bound to the surface of the core structure via their respective first ends, and
wherein the first and second functional groups form an external mosaic of surface domains, each domain comprising a majority of one type of functional group.

27. A particle as recited in claim 26, wherein at least one of the first functional group or the second functional group or the core structure further comprises a temperature sensitive agent.

28. A particle as recited in claim 26, wherein at least one of the first functional group or the second functional group or the core structure further comprises a pH sensitive agent.

29. A particle as recited in claim 26, wherein at least one of the first functional group or the second functional group or the core structure further comprises a light sensitive agent.

30. A particle as recited in claim 26, wherein at least one of the first functional group or the second functional group or the core structure further comprises a conductivity sensitive agent.
